(19) United States
(12) Patent Application Publication
(10) Pub. No.: US 2022/0243208 A1
(43) Pub. Date: Aug. 4, 2022

(54) APTAMERS AGAINST GLIOBLASTOMA

(71) Applicant: SQUADRA Lifesciences, Inc.., Palo Alto, CA (US)

(72) Inventors: Alessadra Affinito, Villaricca (IT); Cristina Quintavalle, Castello di Cisterna (IT); Gerolama Condorelli, Napoli (IT)

(73) Assignee: SQUADRA Lifesciences, Inc.., Palo Alto, CA (US)

(21) Appl. No.: 17/619,206

(22) PCT Filed: May 13, 2020
(86) PCT No.: PCT/IB2020/054523

§ 371 Date: Dec. 14, 2021

(30) Foreign Application Priority Data
May 15, 2019 (IT) 102019000006850

(51) Int. Cl. C12N 15/115; B82Y 5/00

(52) U.S. Cl. CPC C12N 15/115; B82Y 5/00; C12N 2310/16; C12N 2310/141; C12N 2310/3519; C12N 2320/30; C12N 2310/3517

(57) ABSTRACT

Aptamers identified capable of binding glioblastoma cells, and their use in methods of medical treatment and prophylaxis are disclosed.

### FIELD OF THE INVENTION

The present invention relates to nucleic acid compounds and particularly, although not exclusively, to ribonucleic acid compounds, capable of binding glioblastoma stem cells and compositions and methods using the same.

### BACKGROUND

Glioblastoma (GBM) is the most frequent and aggressive primary brain tumor in adults (Louis et al., 2016, Sant et al., 2012). Standard treatments for GBM patients consist of tumor resection, radiotherapy, and chemotherapy with the alkylating agent temozolomide. However, despite advances in surgical and medical neuro-oncology, prognosis for GBM patients remains dismal, with a median survival of 14-15 months (Urbanska et al., 2014). A small population of cancer stem cells (glioblastoma stem cells, GSCs) that retain stem cell properties, including self-renewal and multipotency, have been implicated as responsible for the frequent relapse of GBM and its resistance to conventional therapeutics (Bao et al., 2006, Bovenberg et al., 2013). In contrast to highly proliferating cells from the tumor bulk, this rare quiescent cell population has the potential to reconstitute the intrinsic heterogeneity of the tumor mass and to spread into the brain (Bovenberg et al., 2013, Wang et al., 2013). Therefore, the development of highly specific and safe molecules able to selectively target and eradicate the GSC population represents a timely and important challenge for the treatment of brain tumors.

Aptamers are short, single-stranded oligonucleotides that are high-affinity ligands and potential antagonists of disease-associated proteins (Ellington and Szostak, 1990). The advantages of aptamers are low toxicity, easy penetration in tumors, and, in some cases, ability to cross the bloodbrain barrier (BBB) (Cheng et al., 2013), making them highly promising diagnostic and therapeutic tools and carriers for therapeutic diffusion throughout the tumor area in the intracranial cavity. By adopting an unbiased cell-based variant of the original combinatorial Systematic Evolution of Ligand by EXponential enrichment (SELEX) procedure, it is possible to generate aptamers that target cell-surface binding-specific biomarkers (Ellington and Szostak, 1990, Catuogno et al., 2016, Fitzwater and Polisky, 1996).

### SUMMARY OF THE INVENTION

The present invention provides aptamers identified against glioblastoma cells. The aptamers are nucleic acid compounds. The nucleic acid compound is preferably an oligonucleotide or polynucleotide, preferably single stranded. In some embodiments the nucleic acid compound may be a ribonucleic acid compound, i.e. an RNA. In some embodiments the aptamers preferably bind glioblastoma cells and have inhibitory activity.

The nucleic acid compound may comprise, or consist of, an nucleotide sequence having at least 80% sequence identity to SEQ ID NO:1. SEQ ID NO:1 corresponds to the nucleotide sequence of the A40s aptamer.

In some embodiments, the nucleic acid compound may comprise, or consist of, a nucleotide sequence having at least 80%, 85%, 90%, 95%, 99%, or 100% sequence identity to SEQ ID NO:1.

In some embodiments, the nucleic acid compound comprises, or consists of, a nucleotide sequence having at least 80%, 85%, 90%, 95%, 99%, or 100% sequence identity to SEQ ID NO:1, wherein the nucleotide sequence is preferably capable of binding to a glioblastoma cell, glioblastoma stem cell or to glioblastoma stem cell tumorspheres.

In some embodiments, the nucleic acid compound comprises, or consists of, a nucleotide sequence having at least 80%, 85%, 90%, 95%, 99%, or 100% sequence identity to SEQ ID NO:2. SEQ ID NO:2 corresponds to the nucleotide sequence of the 40 L aptamer. The nucleotide sequence is preferably capable of binding to a glioblastoma cell, glioblastoma stem cell or to glioblastoma stem cell tumorspheres.

In some embodiments, the nucleic acid compound, or nucleotide sequence has a length of 100, 95, 90, 85, 80, 75, 70, 65, 60, 55, 50, 45, 40, 35, 30, 29, 28, 27, 26, 25, or 24 nucleotides or fewer.

In some embodiments, the nucleic acid compound or nucleotide sequence is 30 nucleotides in length.

In some embodiments, the nucleic acid compound or nucleotide sequence is 24 nucleotides in length.

The nucleic acid compound may comprise one or more modified nucleobases, optionally selected from 2′-fluoro (2′-F), 2′-amino (2′-NH2) or 2′-O-methyl (2′-OCH₃). In some embodiments one or all pyrimidines in the nucleic acid compound or nucleotide sequence comprises a 2′ modified nucleobase, optionally selected from 2′-fluoro (2′-F), 2′-amino (2′-NH₂) or 2′-O-methyl (2′-OCH₃).

In some embodiments, the nucleic acid compound has inhibitory activity. Inhibitory activity may include one or more of inhibition of tumour cell proliferation, inhibition of glioblastoma cell proliferation, inhibition of stem cell stemness, inhibition of stem cell growth, inhibition of cell migration, and inhibition of stem cell migration.

Therefore, in some embodiments, the nucleic acid compound may reduce tumour cell proliferation, reduce glioblastoma cell proliferation, inhibit stem cell sternness, inhibit cell growth, inhibit cell migration, and/or inhibit stem cell migration.

In some embodiments, the nucleic acid compound may be capable of being internalised into a cell.

In some embodiments, the nucleic acid compound further comprises a compound moiety attached to said nucleotide sequence. The compound moiety may be a therapeutic moiety or an imaging moiety. It may be a non-nucleic acid moiety, or alternatively a further nucleic acid compound. It may be covalently attached to said nucleotide sequence.

In some embodiments, the nucleic acid compound further comprises a therapeutic moiety attached to said nucleotide sequence, wherein said therapeutic moiety is (i) a nucleic acid moiety, a peptide moiety or a small molecule drug moiety, (ii) an activating nucleic acid moiety or an antisense nucleic acid moiety, and/or (iii) an miRNA, mRNA, saRNA or siRNA moiety, optionally miR-34c. In some embodiments, the therapeutic moiety is an anticancer therapeutic moiety.

In some embodiments, the nucleic acid compound further comprises an imaging moiety attached to said nucleotide sequence, wherein the imaging moiety is a bioluminescent molecule, a photoactive molecule, a metal or a nanoparticle.

The present invention also provides a pharmaceutical composition comprising a nucleic acid compound according to any previous embodiment, and optionally the composition comprises a pharmaceutically acceptable excipient.

The present invention also provides a method of delivering a compound moiety into a cell, optionally a cell in vitro, the method comprising:
(i) contacting a cell with the nucleic acid compound; and
(ii) allowing said nucleic acid compound to bind to a cell and pass into said cell thereby delivering said compound moiety into said cell.

The present invention also provides a method of delivering a compound into a cell, optionally a cell in vitro, the method comprising:
(i) contacting a cell with a compound and the nucleic acid compound; and
(ii) allowing said nucleic acid compound to bind to a cell and pass into said cell thereby delivering said compound into said cell.

The present invention also provides a nucleic acid or pharmaceutical composition according to any embodiment described herein for use in a method of medical treatment or prophylaxis.

In some embodiments, the use of a nucleic acid according to any embodiment described herein is provided for use in the manufacture of a medicament for use in a method of treating or preventing a disease or disorder.

The present invention also provides a method of treating or preventing a disease or disorder, the method comprising administering to a subject in need thereof an effective amount of a nucleic acid compound according to any embodiment described herein, or a composition according to any embodiment described herein.

The method of medical treatment or prophylaxis may be treatment or prophylaxis of cancer, optionally cancer of the peripheral nervous system or central nervous system, such as brain cancer and/or glioblastoma. Methods of medical treatment may further comprise administering an anticancer agent.

The present invention also provides a method, optionally an in vitro method, of detecting a cell, the method comprising:
(i) contacting a cell with the nucleic acid compound according to any embodiment described herein, or a composition according to any embodiment described herein, wherein the nucleic acid compound comprises an imaging moiety;
(ii) allowing said nucleic acid compound to bind to a cell and pass into said cell; and
(iii) detecting said imaging moiety thereby detecting said cell.

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures in which:

FIGS. 1A and B. Charts illustrating GSC aptamer selection. (A) The random regions of all the sequenced aptamers were aligned using Clustal program. Dendrogram shows visual classification of similarity among 100 individual sequences cloned after 16 rounds of selection. (B) The enriched pools from rounds 10, 11, 13, 14, 15, and 16 were sequenced by high throughput sequencing (HTS).

FIG. 2A to D. Charts showing binding of the enriched sequences. Binding was performed with the most enriched sequences at 200 nM on GSC #1 stem (A) or adherent (diff) cells from the same patient (B); Binding with aptamer 40 L was performed at 200 nM on several GSC lines obtained from patients undergoing surgery. Cells grew in suspension (C) or under adherent conditions (D). Representative experiments are shown and results are expressed relative to the background binding detected with the starting pool of sequences used for selection. Vertical bars indicate standard deviation values.

FIG. 3A to D. Charts showing functional inhibition in vitro with 40 L. (A) 20 wells per doses of cells were treated with the aptamer and limiting dilution analysis (LDA) was performed using Extreme Limiting Dilution Analysis (http://bioinf.wehi.edu.au/software/elda). Confidence intervals for stem cell frequency is shown. LDA revealed a reduced stem cell frequency in GSC #83 after 40 L treatment. Estimate stem cell frequency is reported in the graph; bars indicate lower and upper confidence intervals for stem cell frequency as calculated by ELDA software. (B) and (C) 40 L induced a decrease in cell viability and cell migration. Stem cells were incubated with 40 L and cell viability evaluated by MTT assay after 6 days (B); results are presented as mean±SD of three independent experiments. Cell migration was analyzed by a transwell migration assay (C), a representative experiment is shown. Results are expressed relative to the background effect detected with the starting pool of sequences used for selection. Vertical bars indicate standard deviation values. (D) Ability of 40 L to be internalized into GSC. Results are expressed as percentage of the total bound after 30 minutes of incubation. Vertical bars indicate standard deviation values. **, p≤0.01; ****, p≤0.0001.

FIG. 4A to F. A40s characterization and in vitro functional inhibition. (A) 40 L aptamer sequence was shortened in order to have a smaller aptamer with the best properties. Tridimensional shape prediction; the selected portion is shown in the square. Binding assay was performed at 200 nM on #83 cells grown as stem (B) or differentiated (C) cells. Representative experiments are shown and results are expressed relative to the background binding detected with an unrelated aptamer of a similar A40s length. (D) A40s ability to be internalized into GSC. Results are expressed as percentage of the total bound after 30 minutes of incubation. (E) A40s-Alexa488 binding. Immunofluorescence assay was performed by treating #83 stem cells with A40s-Alexa488 or Scrambled-Alexa488 at 500 nM for 30 minutes. All images were captured at the same settings, enabling direct comparison of staining patterns. (F) Stem or differentiated GBM cells were incubated with A40s/miR-34c chimera for 24 and 48 hrs. Relative miR-34c levels were assessed by using qRT-PCR. In (B), (C), (D), and (E) vertical bars indicate standard deviation values.

FIGS. 5A and B. A40s in vitro functional inhibition. 20 wells per doses of cells from GSC #1 (A) or 83 (B) were treated with the A40s aptamer and limiting dilution analyses (LDA) were performed using Extreme Limiting Dilution Analysis (http://bioinf.wehi.edu.au/software/elda). Confidence intervals for stem cell frequency is shown. A40s treatment reduced stem cell frequency in GSCs. Estimate stem cell frequency is reported in the graph; bars indicate lower and upper confidence intervals for stem cell frequency, as calculated by ELDA software. *, p≤0.05; **, p≤0.01.

FIG. 6A to C. A40s in vivo effects. (A) Non-denaturing polyacrylamide gel electrophoresis illustrates A40s stability in 90% human serum. (B) In vivo experiments were performed by inoculating three mice per group on both flanks. In order to asses A40s ability to reach and reduce tumor size, A40s aptamer was intravenously injected. Tumor growth is strongly reduced after A40s treatment compared to scrambled sequence. Correlation coefficient squared (R2) shows a decrease of correlation between weeks and tumor size in A40s treated mice. The arrows indicate the weeks of treatment. Results are presented as mean±SEM; *, p≤0.05. (C) Immuno-histochemistry analysis showing Ki-67 and H&E staining of superfrost slides, using standard methodology.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

### Definitions

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

The abbreviations used herein have their conventional meaning within the chemical and biological arts. The chemical structures and formulae set forth herein are constructed according to the standard rules of chemical valency known in the chemical arts.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & 20 Sons (New York, N.Y. 1994); Sambrook et al., MOLECULAR CLONING, A LABORATORY MANUAL, Cold Springs Harbor Press (Cold Springs Harbor, N Y 1989). Any methods, devices and materials similar or equivalent to those described herein can be used in the practice of this invention. The following definitions are provided to facilitate understanding of certain terms used frequently herein and are not meant to limit the scope of the present disclosure.

“Nucleic acid” refers to deoxyribonucleotides or ribonucleotides and polymers thereof in either single-, double- or multiple-stranded form, or complements thereof. The terms “oligonucleotide” and “polynucleotide” each refers to a linear sequence of nucleotides. The term “nucleotide” typically refers to a single unit of an oligonucleotide or polynucleotide, i.e., a monomer. Nucleotides can be ribonucleotides, deoxyribonucleotides, or modified versions thereof. Examples of oligonucleotides and polynucleotides contemplated herein include single and double stranded DNA, single and double stranded RNA (including siRNA), and hybrid molecules having mixtures of single and double stranded DNA and RNA. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like. In preferred embodiments, nucleic acids are linear, non-branched, although they may fold to adopt secondary structure motifs, e.g. a stem loop.

Nucleic acids, including nucleic acids with a phosphothioate backbone can include one or more reactive moieties. As used herein, the term reactive moiety includes any group capable of reacting with another molecule, e.g., a nucleic acid or polypeptide through covalent, noncovalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, Oligonucleotides and Analogues: A Practical Approach, Oxford University Press); and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA)), including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may′ be made. In embodiments, the internucleotide linkages in DNA are phosphodiester, phosphodiester derivatives, or a combination of both.

The words “complementary” or “complementarity” refer to the ability of a nucleic acid in a polynucleotide to form a base pair with another nucleic acid in a second polynucleotide. For example, the sequence A-G-T is complementary to the sequence T-C-A. Complementarity may be partial, in which only some of the nucleic acids match according to base pairing, or complete, where all the nucleic acids match according to base pairing.

The term “probe” or “primer”, as used herein, is defined to be one or more nucleic acid fragments whose specific hybridization to a sample can be detected. A probe or primer can be of any length depending on the particular technique it will be used for. For example, PCR primers are generally between 10 and 40 nucleotides in length, while nucleic acid probes for, e.g., a Southern blot, can be more than a hundred nucleotides in length. The probe may be unlabeled or labeled as described below so that its binding to the target or sample can be detected. The probe can be produced from a source of nucleic acids from one or more particular (preselected) portions of a chromosome, e.g., one or more clones, an isolated whole chromosome or chromosome fragment, or a collection of polymerase chain reaction (PCR) amplification products. The length and complexity of the nucleic acid fixed onto the target element is not critical to the invention. One of skill can adjust these factors to provide optimum hybridization and signal production for a given hybridization procedure, and to provide the required resolution among different genes or genomic locations.

The probe may also be isolated nucleic acids immobilized on a solid surface (e.g., nitrocellulose, glass, quartz, fused silica slides), as in an array. In some embodiments, the probe may be a member of an array of nucleic acids as described, for instance, in WO 96/17958. Techniques capable of producing high density arrays can also be used for this purpose (see, e.g., Fodor (1991) Science 767-773; Johnston (1998) Curr. Biol. 8: R171-R174; Schummer (1997) Biotechniques 23:1087-1092; Kern (1997) Biotechniques 23:120-124; U.S. Pat. No. 5,143,854).

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules (e.g., siRNA) may be detected by standard PCR or Northern blot methods well known in the art. See, Sambrook et al., 1989 Molecular Cloning: A Laboratory Manual, 18.1-18.88.

The term “aptamer” as provided herein refers to oligonucleotides (e.g. short oligonucleotides or deoxyribonucleotides), that bind (e.g. with high affinity and specificity) to a target molecule, typically a protein, peptide, or small molecule. Aptamers typically have defined secondary or tertiary structure owing to their propensity to form complementary base pairs and, thus, are often able to fold into diverse and intricate molecular structures. The three-dimensional structures are essential for aptamer binding affinity and specificity, and specific three-dimensional interactions drives the formation of aptamer-target complexes. Aptamers can be selected in vitro from very large libraries of randomized sequences by the process of systemic evolution of ligands by exponential enrichment (SELEX as described in Ellington A D, Szostak J W (1990) In vitro selection of RNA molecules that bind specific ligands. Nature 346:818-822; Tuerk C, Gold L (1990) Systematic evolution of ligands by exponential enrichment: RNA ligands to bacteriophage T4 DNA polymerase. Science 249:505-510) or by developing SOMAmers (slow off-rate modified aptamers) (Gold L et al. (2010) Aptamer-based multiplexed proteomic technology for biomarker discovery. PLoS ONE 5(12):e15004). Applying the SELEX and the SOMAmer technology includes for instance adding functional groups that mimic amino acid side chains to expand the aptamer's chemical diversity. As a result high affinity aptamers for almost any protein target are enriched and identified. Aptamers exhibit many desirable properties for targeted drug delivery, such as ease of selection and synthesis, high binding affinity and specificity, flexible structure, low immunogenicity, and versatile synthetic accessibility. To date, a variety of anti-cancer agents (e.g. chemotherapy drugs, toxins, and siRNAs) have been successfully delivered to cancer cells in vitro using aptamers.

An “antisense nucleic acid” as referred to herein is a nucleic acid (e.g. DNA or RNA molecule) that is complementary to at least a portion of a specific target nucleic acid (e.g. an mRNA translatable into a protein) and is capable of reducing transcription of the target nucleic acid (e.g. mRNA from DNA) or reducing the translation of the target nucleic acid (e.g. mRNA) or altering transcript splicing (e.g. single stranded morpholino oligo). See, e.g., Weintraub, Scientific American; 262:40 (1990). Typically, synthetic antisense nucleic acids (e.g. oligonucleotides) are generally between 15 and 25 bases in length. Thus, antisense nucleic acids are capable of hybridizing to (e.g. selectively hybridizing to) a target nucleic acid (e.g. target mRNA). In embodiments, the antisense nucleic acid hybridizes to the target nucleic acid sequence (e.g. mRNA) under stringent hybridization conditions. In embodiments, the antisense nucleic acid hybridizes to the target nucleic acid (e.g. mRNA) under moderately stringent hybridization conditions. Antisense nucleic acids may comprise naturally occurring nucleotides or modified nucleotides such as, e.g., phosphorothioate, methylphosphonate, and -anomeric sugar-phosphate, backbone modified nucleotides.

In the cell, the antisense nucleic acids hybridize to the corresponding mRNA, forming a double-stranded molecule. The antisense nucleic acids interfere with the translation of the mRNA, since the cell will not translate an mRNA that is double-stranded. The use of antisense methods to inhibit the in vitro translation of genes is well known in the art (Marcus-Sakura, Anal. Biochem., 172:289 (1988)). Further, antisense molecules which bind directly to the DNA may be used. Antisense nucleic acids may be single or double stranded nucleic acids. Non-limiting examples of antisense nucleic acids include siRNAs (including their derivatives or pre-cursors, such as nucleotide analogs), short hairpin RNAs (shRNA), micro RNAs (miRNA), saRNAs (small activating RNAs) and small nucleolar RNAs (snoRNA) or certain of their derivatives or pre-cursors.

A “siRNA,” “small interfering RNA,” “small RNA,” or “RNAi” as provided herein, refers to a nucleic acid that forms a double stranded RNA, which double stranded RNA has the ability to reduce or inhibit expression of a gene or target gene when expressed in the same cell as the gene or target gene. The complementary portions of the nucleic acid that hybridize to form the double stranded molecule typically have substantial or complete identity. In one embodiment, a siRNA or RNAi is a nucleic acid that has substantial or complete identity to a target gene and forms a double stranded siRNA. In embodiments, the siRNA inhibits gene expression by interacting with a complementary cellular mRNA thereby interfering with the expression of the complementary mRNA. Typically, the nucleic acid is at least about 15-50 nucleotides in length (e.g., each complementary sequence of the double stranded siRNA is 15-50 nucleotides in length, and the double stranded siRNA is about 15-50 base pairs in length). In other embodiments, the length is 20-30 base nucleotides, preferably about 20-25 or about 24-30 nucleotides in length, e.g., 20, 21, 22, 23, 24, 25, 26, 27, 28, 29, or 30 nucleotides in length.

A “snRNA,” or “small activating RNA” as provided herein refers to a nucleic acid that forms a double stranded RNA, which double stranded RNA has the ability to increase or activate expression of a gene or target gene when expressed in the same cell as the gene or target gene. The complementary portions of the nucleic acid that hybridize to form the double stranded molecule typically have substantial or complete identity. In one embodiment, a saRNA is a nucleic acid that has substantial or complete identity to a target gene and forms a double stranded saRNA. Typically, the nucleic acid is at least about 15-50 nucleotides in length (e.g., each complementary sequence of the double stranded saRNA is 15-50 nucleotides in length, and the double stranded saRNA is about 15-50 base pairs in length). In other embodiments, the length is 20-30 base nucleotides, preferably about 20-25 or about 24-29 nucleotides in length, e.g., 20, 21, 22, 23, 24, 25, 26, 27, 28, 29, or 30 nucleotides in length.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “purified” denotes that a nucleic acid or protein gives rise to essentially one band in an electrophoretic gel. In some embodiments, the nucleic acid or protein is at least 50% pure, optionally at least 65% pure, optionally at least 75% pure, optionally at least 85% pure, optionally at least 95% pure, and optionally at least 99% pure.

The term “isolated” may also refer to a cell or sample cells. An isolated cell or sample cells are a single cell type that is substantially free of many of the components which normally accompany the cells when they are in their native state or when they are initially removed from their native state. In certain embodiments, an isolated cell sample retains those components from its natural state that are required to maintain the cell in a desired state. In some embodiments, an isolated (e.g. purified, separated) cell or isolated cells, are cells that are substantially the only cell type in a sample. A purified cell sample may contain at least 60%, 70%, 75%, 80%, 85%, 90%, 95%, 96%, 97%, 98%, 99%, or 100% of one type of cell. An isolated cell sample may be obtained through the use of a cell marker or a combination of cell markers, either of which is unique to one cell type in an unpurified cell sample. In some embodiments, the cells are isolated through the use of a cell sorter. In some embodiments, antibodies against cell proteins are used to isolate cells.

As used herein, the term “conjugate” refers to the association between atoms or molecules. The association can be direct or indirect. For example, a conjugate between a nucleic acid (e.g., ribonucleic acid) and a compound moiety as provided herein can be direct, e.g., by covalent bond, or indirect, e.g., by non-covalent bond. Optionally, conjugates are formed using conjugate chemistry including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. Thus, the nucleic acid acids can be attached to a compound moiety through its backbone. Optionally, the nucleic acid includes one or more reactive moieties, e.g., an amino acid reactive moiety, that facilitates the interaction of the nucleic acid with the compound moiety.

Useful reactive moieties or functional groups used for conjugate chemistries herein include, for example:

(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;

(b) hydroxyl groups which can be converted to esters, ethers, aldehydes, etc;

(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;

(d) dienophile groups which are capable of participating in Diels-Alder reactions such as, for example, maleimido groups;

(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;

(f) sulfonyl halide groups for subsequent reaction with amines, for example, to form sulfonamides;

(g) thiol groups, which can be converted to disulfides, reacted with acyl halides, or bonded to metals such as gold;

(h) amine or sulfhydryl groups, which can be, for example, acylated, alkylated or oxidized;

(i) alkenes, which can undergo, for example, cycloadditions, acylation, Michael addition, etc;

(j) epoxides, which can react with, for example, amines and hydroxyl compounds;

(k) phosphoramidites and other standard functional groups useful in nucleic acid synthesis;

(l) metal silicon oxide bonding;

(m) metal bonding to reactive phosphorus groups (e.g. phosphines) to form, for example, phosphate diester bonds; and

(n) sulfones, for example, vinyl sulfone.

The reactive functional groups can be chosen such that they do not participate in, or interfere with, the chemical stability of the proteins described herein. By way of example, the nucleic acids can include a vinyl sulfone or other reactive moiety. Optionally, the nucleic acids can include a reactive moiety having the formula S—S—R. R can be, for example, a protecting group. Optionally, R is hexanol. As used herein, the term hexanol includes compounds with the formula C₆H₁₃OH and includes, 1-hexanol, 2-hexanol, 3-hexanol, 2-methyl-1-pentanol, 3-methyl-1-pentanol, 4-methyl-1-pentanol, 2-methyl-2-pentanol, 3-methyl-2-pentanol, 4-methyl-2-pentanol, 2-methyl-3-pentanol, 3-methyl-3-pentanol, 2,2-dimethyl-1-butanol, 2,3-dimethyl-1-butanol, 3,3-dimethyl-1-butanol, 2,3-dimethyl-2-butanol, 3,3-dimethyl-2-butanol, and 2-ethyl-1-butanol. Optionally, R is 1-hexanol.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In some embodiments, about means the specified value.

The terms “protein”, “peptide”, and “polypeptide” are used interchangeably to denote an amino acid polymer or a set of two or more interacting or bound amino acid polymers. The terms apply to amino acid polymers in which one or more than one amino acid residue is an artificial chemical mimetic of a corresponding naturally occurring amino acid, as well as to naturally occurring amino acid polymers and non-naturally occurring amino acid polymers.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

For specific proteins described herein, the named protein includes any of the protein's naturally occurring forms, variants or homologs that maintain the protein transcription factor activity (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference, homolog or functional fragment thereof.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., Spodoptera) and human cells.

The term “glioblastoma cell” refers to any cell which is part of a glioblastoma and forms part of the tumor. The term “glioblastoma stem cell” or “GSC” refers to a glioblastoma cell which retains stem cell properties. GSCs can be isolated through mechanical dissociation of glioblastoma tumor specimens, as used in the examples of the current application and as previously described (Ricci-Vitiani et al., 2010, Pallini et al., 2008). The term “cancer stem cell” or “CSC” refers to cancer cells (found within tumors or hematological cancers) that possess characteristics associated with normal stem cells, specifically the ability to give rise to all cell types found in a particular cancer sample. CSCs have been identified in various solid tumors. Commonly, markers specific for normal stem cells are used for isolating CSCs from solid and hematological tumors. Markers most frequently used for CSC isolation include: CD133 (also known as PROM1), CD44, ALDH1A1, CD34, CD24 and EpCAM (epithelial cell adhesion molecule, also known as epithelial specific antigen, ESA).

The term “blood-brain barrier” refers to a highly selective semipermeable membrane barrier that separates the circulating blood from the brain and extracellular fluid in the central nervous system. The barrier provides tight regulation of the movement of ions, molecules and cells between the blood and the brain, see e.g. Daneman and Prat, Cold Spring Harb Perspect Biol. 2015; 7(1):a020412. Many therapeutic molecules are generally excluded from transport from blood to brain due to their negligible permeability over the brain capillary endothelial wall.

“Anti-cancer agent” is used in accordance with its plain ordinary meaning and refers to a composition (e.g. compound, drug, antagonist, inhibitor, modulator) having antineoplastic properties or the ability to inhibit the growth or proliferation of cells. In embodiments, an anticancer agent is a chemotherapeutic. In embodiments, an anti-cancer agent is an agent identified herein having utility in methods of treating cancer. In embodiments, an anti-cancer agent is an agent approved by the FDA or similar regulatory agency of a country other than the USA, for treating cancer. Examples of anti-cancer agents include, but are not limited to, MEK (e.g. MEK1, MEK2, or MEK1 and MEK2) inhibitors (e.g. XL518, CT-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, melphalan), ethylenimine and methylmelamines (e.g., hexamethlymelamine, thiotepa), alkyl sulfonates (e.g., busulfan), nitrosoureas (e.g., carmustine, lomustine, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP 16), etoposide phosphate, teniposide, etc.), anti tumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin), anthracenedione (e.g., mitoxantrone), substituted urea (e.g., hydroxyurea), methyl hydrazine derivative (e.g., procarbazine), or adrenocortical suppressant (e.g., mitotane, aminoglutethimide), epipodophyllotoxins (e.g., etoposide).

Further examples of anti-cancer agents include, but are not limited to, antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002), mTOR inhibitors, antibodies (e.g., rituxan), 5-aza-2′-deoxycytidine, doxorubicin, vincristine, etoposide, gemcitabine, imatinib (Gleevec®), geldanamycin, 17-N-Allylamino-17-Demethoxygeldanamycin (17-AAG), bortezomib, trastuzumab, anastrozole; angiogenesis inhibitors; antiandrogen, antiestrogen; antisense oligonucleotides; apoptosis gene modulators; apoptosis regulators; arginine deaminase; BCR/ABL antagonists; beta lactam derivatives; bFGF inhibitor; bicalutamide; camptothecin derivatives; casein kinase inhibitors (ICOS); clomifene analogues; cytarabine dacliximab; dexamethasone; estrogen agonists; estrogen antagonists; etanidazole; etoposide phosphate; exemestane; fadrozole; finasteride; fludarabine; fluorodaunorunicin hydrochloride; gadolinium texaphyrin; gallium nitrate; gelatinase inhibitors; gemcitabine; glutathione inhibitors; hepsulfam; immunostimulant peptides; insulin-like growth factor-I receptor inhibitor; interferon agonists; interferons; interleukins; letrozole; leukemia inhibiting factor; leukocyte alpha interferon; leuprolide+estrogen+progesterone; leuprorelin; matrilysin inhibitors; matrix metalloproteinase inhibitors; MIF inhibitor; mifepristone; mismatched double stranded RNA; monoclonal antibody; mycobacterial cell wall extract; nitric oxide modulators; oxaliplatin; panomifene; pentrozole; phosphatase inhibitors; plasminogen activator inhibitor; platinum complex; platinum compounds; prednisone; proteasome inhibitors; protein A-based immune modulator; protein kinase C inhibitor; protein kinase C inhibitors, protein tyrosine phosphatase inhibitors; purine nucleoside phosphorylase inhibitors; ras farnesyl protein transferase inhibitors; ras inhibitors; ras-GAP inhibitor; ribozymes; signal transduction inhibitors; signal transduction modulators; single chain antigen-binding protein; stem cell inhibitor; stem-cell division inhibitors; stromelysin inhibitors; synthetic glycosaminoglycans; tamoxifen methiodide; telomerase inhibitors; thyroid stimulating hormone; translation inhibitors; tyrosine kinase inhibitors; urokinase receptor antagonists; steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guerin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-Pseudomonas exotoxin conjugate, etc.), radioimmunotherapy (e.g., anti-CD20 monoclonal antibody conjugated to ¹¹¹In, ⁹⁰Y, or ¹³¹I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Tressa™), erlotinib (Tarceva™), cetuximab (Erbitux™), lapatinib (Tykerb™), panitumumab (Vectibix™), vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, or the like.

“Chemotherapeutic” or “chemotherapeutic agent” is used in accordance with its plain ordinary meaning and refers to a chemical composition or compound having antineoplastic properties or the ability to inhibit the growth or proliferation of cells.

Additionally, the nucleic acid compound described herein can be co-administered with or covalently attached to conventional immunotherapeutic agents including, but not limited to, immunostimulants (e.g., Bacillus Calmette-Guerin (BCG), levamisole, interleukin-2, alphainterferon, etc.), monoclonal antibodies (e.g., anti-CD20, anti-HER2, anti-CD52, anti-HLA-DR, anti-PD-1 and anti-VEGF monoclonal antibodies), immunotoxins (e.g., anti-CD33 monoclonal antibody-calicheamicin conjugate, anti-CD22 monoclonal antibody-Pseudomonas exotoxin conjugate, etc.), and radioimmunotherapy (e.g., anti-CD20 monoclonal antibody conjugated to ¹¹¹In, ⁹⁰Y, or ¹³¹I, etc.).

In a further embodiment, the nucleic acid compounds described herein can be co-administered with conventional radiotherapeutic agents including, but not limited to, radionuclides such as ⁴⁷Sc, ⁶⁴Cu, ⁶⁷Cu, ⁸⁹Sr, ⁸⁶Y, ⁸⁷Y, ⁹⁰Y, ¹⁰⁵Rh, ¹¹¹Ag, ¹¹¹In, ¹¹⁷Sn, ¹⁴⁹Pm, ¹⁵³Sm, ¹⁶⁶Ho, ¹⁷⁷Lu, ¹⁸⁶Re, ¹⁸⁸Re, ²¹¹At, and ²¹²Bi, optionally conjugated to antibodies directed against tumor antigens.

The term “sample” includes sections of tissues such as biopsy and autopsy samples, and frozen sections taken for histological purposes. Such samples include blood and blood fractions or products (e.g., bone marrow, serum, plasma, platelets, red blood cells, and the like), sputum, tissue, cultured cells (e.g., primary cultures, explants, and transformed cells), stool, urine, other biological fluids (e.g., prostatic fluid, gastric fluid, intestinal fluid, renal fluid, lung fluid, cerebrospinal fluid, and the like), etc. A sample is typically obtained from a “subject” such as a eukaryotic organism, most preferably a mammal such as a primate, e.g., chimpanzee or human; cow; dog; cat; a rodent, e.g., guinea pig, rat, mouse; rabbit; or a bird; reptile; or fish. In some embodiments, the sample is obtained from a human.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

“Disease” or “condition” refer to a state of being or health status of a patient or subject capable of being treated with a compound, pharmaceutical composition, or method provided herein. In embodiments, the disease is cancer (e.g. glioblastoma). As used herein, the term “cancer” refers to all types of cancer, neoplasm or malignant tumors found in mammals, including leukemia, lymphoma, carcinomas and sarcomas. Exemplary cancers that may be treated with a compound, pharmaceutical composition, or method provided herein include glioblastoma.

A cancer may be any unwanted cell proliferation (or any disease manifesting itself by unwanted cell proliferation), neoplasm or tumor or increased risk of or predisposition to the unwanted cell proliferation, neoplasm or tumor. The cancer may be benign or malignant and may be primary or secondary (metastatic). A neoplasm or tumor may be any abnormal growth or proliferation of cells and may be located in any tissue. Examples of tissues include the adrenal gland, adrenal medulla, anus, appendix, bladder, blood, bone, bone marrow, brain, breast, cecum, central nervous system (including or excluding the brain) cerebellum, cervix, colon, duodenum, endometrium, epithelial cells (e.g. renal epithelia), gallbladder, oesophagus, glial cells, heart, ileum, jejunum, kidney, lacrimal glad, larynx, liver, lung, lymph, lymph node, lymphoblast, maxilla, mediastinum, mesentery, myometrium, nasopharynx, omentume, oral cavity, ovary, pancreas, parotid gland, peripheral nervous system, peritoneum, pleura, prostate, salivary gland, sigmoid colon, skin, small intestine, soft tissues, spleen, stomach, testis, thymus, thyroid gland, tongue, tonsil, trachea, uterus, vulva, white blood cells.

Tumors to be treated may be nervous or non-nervous system tumors. Nervous system tumors may originate either in the central or peripheral nervous system, e.g. glioma, glioblastoma, medulloblastoma, meningioma, neurofibroma, ependymoma, Schwannoma, neurofibrosarcoma, astrocytoma and oligodendroglioma. Nervous system tumors may be primary or secondary. A nervous syatem tumor may be a primary or secondary brain cancer or tumor, i.e. occurring in the brain. Non-nervous system cancers/tumors may originate in any other non-nervous tissue, examples include melanoma, mesothelioma, lymphoma, myeloma, leukemia, Non-Hodgkin's lymphoma (NHL), Hodgkin's lymphoma, chronic myelogenous leukemia (CML), acute myeloid leukemia (AML), myelodysplastic syndrome (MDS), cutaneous T-cell lymphoma (CTCL), chronic lymphocytic leukemia (CLL), hepatoma, epidermoid carcinoma, prostate carcinoma, breast cancer, lung cancer, colon cancer, ovarian cancer, pancreatic cancer, thymic carcinoma, NSCLC, haematologic cancer and sarcoma.

As used herein, the terms “metastasis,” “metastatic,” and “metastatic cancer” can be used interchangeably and refer to the spread of a proliferative disease or disorder, e.g., cancer, from one organ or another non-adjacent organ or body part. Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The term “associated” or “associated with” in the context of a substance or substance activity or function associated with a disease (e.g., diabetes, cancer (e.g. prostate cancer, renal cancer, metastatic cancer, melanoma, castration-resistant prostate cancer, breast cancer, triple negative breast cancer, glioblastoma, ovarian cancer, lung cancer, squamous cell carcinoma (e.g., head, neck, or esophagus), colorectal cancer, leukemia, acute myeloid leukemia, lymphoma, B cell lymphoma, or multiple myeloma)) means that the disease (e.g., diabetes, cancer (e.g. prostate cancer, renal cancer, metastatic cancer, melanoma, castration-resistant prostate cancer, breast cancer, triple negative breast cancer, glioblastoma, ovarian cancer, lung cancer, squamous cell carcinoma (e.g., head, neck, or esophagus), colorectal cancer, leukemia, acute myeloid leukemia, lymphoma, B cell lymphoma, or multiple myeloma) or viral disease (e.g., HN infection associated disease)) is caused by (in whole or in part), or a symptom of the disease is caused by (in whole or in part) the substance or substance activity or function.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g. by using a method as described herein), results in reduction of the disease or one or more disease symptoms.

“Contacting” is used in accordance with its plain ordinary meaning and refers to the process of allowing at least two distinct species (e.g. chemical compounds including biomolecules, or cells) to become sufficiently proximal to react, interact or physically touch. It should be appreciated, however, that the resulting reaction product can be produced directly from a reaction between the added reagents or from an intermediate from one or more of the added reagents which can be produced in the reaction mixture. Contacting may include allowing two species to react, interact, or physically touch, wherein the two species may be a nucleic acid compound as described herein and a cell (e.g., cancer cell).

### Glioblastoma

Glioblastoma, or astrocytoma WHO grade IV, is the most fatal primary brain cancer found in humans, and most glioblastomas manifest rapidly de novo, without recognizable precursor lesions (Bleeker et al., 2012). The standard treatment for newly diagnosed glioblastoma patients is gross total removal, if possible, followed by the combination of the alkylating cytostatic drug temozolomide (TMZ) and RT (Stupp et al., 2005, Stupp et al., 2009). Clinically, gliomas are divided into four grades and the most aggressive of these, grade IV astrocytoma or glioblastoma, is also the most common in humans (Kleihues and Cavanee, 2000).

One of the first steps in tumor invasions is migration. GBM cells have the ability to infiltrate and disrupt physical barriers such as basement membranes, extracellular matrix and cell junctions (Rodrigues Alves et al., 2011).

The cellular origin of glioblastoma is currently unknown. Because of the similarities in immunostaining of glial cells and glioblastoma, it has long been assumed that gliomas such as glioblastoma originate from glial type cells. More recent studies suggest that astrocytes, oligodendrocyte progenitor cells and neural stem cells could all serve as the cell of origin (Zong et al., 2012, Zong et al., 2015).

Glioblastoma tumors are characterized by the presence of small areas of necrotizing tissue that are surrounded by anaplastic cells. This characteristic, as well as the presence of hyperplastic blood vessels, differentiates the tumor from Grade 3 astrocytomas, which do not have these features. Malignant cells carried in the CSF may spread (rarely) to the spinal cord or cause meningeal gliomatosis. However, metastasis of GBM beyond the central nervous system is unusual.

The tumor may take on a variety of appearances, depending on the amount of hemorrhage, necrosis, or its age. A CT scan will usually show an inhomogeneous mass with a hypodense center and a variable ring of enhancement surrounded by edema. Mass effect from the tumor and edema may compress the ventricles and cause hydrocephalus.

A sub-population of cells within glioblastomas with stem-like properties may be the source of tumors these cells (GSCs) are highly resistant to current cancer treatments. These cancer therapies, while killing the majority of tumor cells, ultimately fail in glioblastoma treatment because they do not eliminate GSCs, which survive to regenerate new tumors (Rodrigues Alves et al., 2011). These GSCs reside in a niche around arterioles, protecting these cells against therapy by maintaining a relatively hypoxic environment (Hira et al., 2015).

These GSCs retain stem cell properties, including self-renewal and multipotency (Bao et al., 2006, Bovenberg et al., 2013). In contrast to highly proliferating cells from the tumor bulk, this rare quiescent cell population has the potential to reconstitute the intrinsic heterogeneity of the tumor mass and to spread into the brain (Bovenberg et al., 2013, Wang et al., 2013). Therefore, the development of highly specific and safe molecules able to selectively target and eradicate the GSC population represents a timely and important challenge for the treatment of brain tumors.

### Nucleic Acid Compounds

The present invention provides nucleic acid compounds that are inter alia capable of binding glioblastoma stem cells (GSCs). In some cases, the nucleic acid compounds are internalised into the cell. In various embodiments, the nucleic acid compounds provided herein comprise a payload, such as a therapeutic or diagnostic molecule, and thus facilitate targeted delivery of the payload to GSCs. The nucleic acid compounds and the payload may be internalised into GSCs, thus providing an efficient mechanism for targeted intracellular delivery.

The three-dimensional structure of a nucleic acid compound, e.g. an aptamer, is essential for determining binding affinity and specificity. Thus, one cannot truncate a nucleic acid compound with the absolute expectation that it will retain its ability to bind the same target. Predicting functional truncated aptamer sequences is not a trivial exercise.

In some aspects and embodiments the nucleic acid compound is a ribonucleic acid compound, and the nucleotide sequence is an RNA.

In some aspects, the present invention provides a nucleic acid compound comprising, or consisting of, an nucleotide sequence having at least 80% sequence identity to SEQ ID NO:1. In any embodiment provided herein, the nucleic acid compound may be a ribonucleic acid compound.

In some embodiments the nucleotide sequence has at least 85%, at least 87%, at least 90%, at least 91%, at least 92%, at least 93%, at least 94%, at least 95%, at least 96%, at least 97%, at least 98% or at least 99% sequence identity to SEQ ID NO:1. In some embodiments, the nucleotide sequence has at least 90% sequence identity to SEQ ID NO:1. In some embodiments the nucleotide sequence has 80%, 85%, 87%, 90%, 91%, 92%, 93%, 94%, 95%, 96%, 97%, 98%, 99% or 100% sequence identity to SEQ ID NO:1. In some embodiments, the RNA sequence has 100% sequence identity to SEQ ID NO:1. In some embodiments, the nucleotide sequence comprises or consists of SEQ ID NO:1.

In some cases the nucleotide sequence has at least 80% sequence identity to a nucleic acid that hybridises to SEQ ID NO:1. In some cases the nucleotide sequence has at least 85%, at least 87%, at least 90%, at least 91%, at least 92%, at least 93%, at least 94%, at least 95%, at least 96%, at least 97%, at least 98%, at least 99%, or at least 100% sequence identity to a nucleic acid that hybridises to SEQ ID NO:1.

In some aspects, the present invention provides a nucleic acid compound comprising, or consisting of, an nucleotide sequence having at least 80% sequence identity to SEQ ID NO:2. In any embodiment provided herein, the nucleic acid compound may be a ribonucleic acid compound.

In some embodiments the nucleotide sequence has at least 85%, at least 87%, at least 90%, at least 91%, at least 92%, at least 93%, at least 94%, at least 95%, at least 96%, at least 97%, at least 98% or at least 99% sequence identity to SEQ ID NO:2. In some embodiments, the nucleotide sequence has at least 90% sequence identity to SEQ ID NO:2. In some embodiments the nucleotide sequence has 80%, 85%, 87%, 90%, 91%, 92%, 93%, 94%, 95%, 96%, 97%, 98%, 99% or 100% sequence identity to SEQ ID NO:2. In some embodiments, the nucleotide sequence has 100% sequence identity to SEQ ID NO:2. In some embodiments, the nucleotide sequence comprises or consists of SEQ ID NO:2.

In some embodiments, the nucleotide sequence is capable of binding to a glioblastoma stem cell (GSC). In some embodiments, the nucleotide sequence binds to a glioblastoma cell, a cancer stem cell (CSC), or a glioblastoma stem cell (GSC). In some embodiments, the nucleic acid compound is capable of being internalised into a cell.

In some cases the nucleotide sequence has at least 80% sequence identity to a nucleic acid that hybridises to SEQ ID NO:2. In some cases the nucleotide sequence has at least 85%, at least 87%, at least 90%, at least 91%, at least 92%, at least 93%, at least 94%, at least 95%, at least 96%, at least 97%, at least 98%, at least 99%, or at least 100% sequence identity to a nucleic acid that hybridises to SEQ ID NO:2.

In some embodiments a nucleotide sequence provided herein has a length of 100 nucleotides or fewer, 95 nucleotides or fewer, 90 nucleotides or fewer, 85 nucleotides or fewer, 80 nucleotides or fewer, 75 nucleotides or fewer, 70 nucleotides or fewer, 65 nucleotides or fewer, 60 nucleotides or fewer, 55 nucleotides or fewer, 50 nucleotides or fewer, 45 nucleotides or fewer, 40 nucleotides or fewer, 35 nucleotides or fewer, 30 nucleotides or fewer, 29 nucleotides or fewer, 28 nucleotides or fewer, 27 nucleotides or fewer, 26 nucleotides or fewer, or 25 nucleotides or fewer.

In some embodiments the nucleotide sequence is between 24 and 90, 24 and 85, 24 and 80, 24 and 75, 24 and 70, 24 and 65, 24 and 60, 24 and 55, 24 and 50, 24 and 45, 24 and 40, 24 and 35, or 24 and 30 nucleotides in length.

In some embodiments the nucleotide sequence is between 30 and 90, 30 and 85, 30 and 80, 30 and 75, 30 and 70, 30 and 65, 30 and 60, 30 and 55, 30 and 50, 30 and 45, 30 and 40, or 30 and 35 nucleotides in length.

In some embodiments the nucleotide sequence is 24, 25, 26, 27, 28, 29, 30, 31, 32, 33, 34, 35, 40, 45, 50, 55, 60, 65, 70, 75, 80, 85, 90, 95, Or 100 nucleotides in length.

In some embodiments, the nucleotide sequence has at least 80%, 85%, 90%, 95%, or 99% sequence identity to SEQ ID NO:1 and has a length of 50 nucleotides or fewer, 45 nucleotides or fewer, 40 nucleotides or fewer, 35 nucleotides or fewer, 30 nucleotides or fewer, or 25 nucleotides or fewer.

In some embodiments, the nucleotide sequence comprises or consists SEQ ID NO:1.

In some cases, an nucleotide sequence provided herein differs by 1, 2, 3, or 4 nucleotides compared to SEQ ID NO:1.

In some embodiments, the nucleotide sequence comprises or consists SEQ ID NO:2.

In some cases, a nucleotide sequence provided herein differs by 1, 2, 3, or 4 nucleotides compared to SEQ ID NO:2.

In some cases, the nucleotide sequence comprises a loop structure. In some cases, the nucleotide sequence comprises a stem-loop structure. In some cases, the nucleotide sequence comprises intramolecular base pairing.

The term “capable of being internalised into a cell” as used herein refers to the ability of a nucleic molecule of the present invention to be transported from the outside of a cell into a cell. This may be performed by cellular mechanisms such as endocytosis or phagocytosis. In some cases, the nucleic acids are internalised after they bind to the cell.

In any embodiment provided herein, the nucleotide sequence comprises ribonucleotide residues, i.e. may be an RNA. In some embodiments, the nucleotide sequence may comprise one or more deoxyribonucleotide residues, i.e. may be a DNA. That is, in some cases, the nucleotide sequence comprises one or more residues selected from deoxyadenosine monophosphate (dAMP), deoxyguanosine monophosphate (dGMP), thymidine monophosphate/deoxythymidine monophosphate (TMP/dTMP) and deoxycytidine monophosphate (dCMP). In some cases, one or more uridine monophosphate (UMP) residues in the nucleotide sequence are substituted for deoxythymidine monophosphate (TMP/dTMP) residues.

In some cases, the nucleic acid compounds described herein comprise one or more modified nucleobases. For example, the nucleic acid compounds may comprise one or more ribo/deoxyribonucleobases modified with a fluoro (F), amino (NH2) or O-methyl (OCH3) group. In some cases, the nucleobases are modified at the 2′ position, the 3′ position, the 5′ position or the 6′ position. In some cases, the nucleic acid compounds may comprise one or more 2′-aminopyrimidines, 2′-fluoropyrimidines, 2′-O-methyl nucleotides and/or ‘locked’ nucleotides (LNA) (see e.g. Lin, Y et al., Nucleic Acids Res. 1994 22, 5229-5234 (1994); Ruckman, J. et al. J. Biol. Chem. 1998 273, 20556-20567; Burmeister, P E et al., Chem. Biol. 2005 12, 25-33; Kuwahara, M. & Obika, S. Artif. DNA PNA XNA 2013 4, 39-48; Veedu, R. N. & Wengel, J. Mol. Biosyst. 2009 5,787-792). In some cases, the nucleic acid compounds comprise one or more L-form nucleic acids (see e.g. Maasch, C et al., Nucleic Acids Symp. Ser. (Oxf.) 2008 52, 61-62). Other suitable nucleic acid modifications will be apparent to those skilled in the art (see, e.g. Ni S et al., Int. J. Mol. Sci 2017 18, 1683, hereby incorporated by reference in its entirety).

In some embodiments, nucleotides comprise 2′ modified ribo/deoxyribo nucleobases, with a modification selected from a 2′-fluoro (F), 2′-amino (NH₂) or 2′-O-methyl (OCH₃) group.

In some embodiments, nucleotides corresponding to positions 1, 2, 3, 5, 6, 8, 9, 10, 13, 20, 21, 22, 24, and 27 of SEQ ID NO:1 comprise ribo/deoxyribo nucleobases modified with a 2′-fluoro (F), amino (NH₂) or O-methyl (OCH₃) group.

In some embodiments, nucleotides corresponding to positions 1, 2, 3, 5, 6, 8, 9, 10, 13, 20, 21, 22, 24, and 27 of SEQ ID NO:1 comprise ribo/deoxyribo nucleobases modified with a 2′-fluoro (F) group.

In some embodiments, nucleotides corresponding to positions 1, 2, 3, 5, 6, 8, 9, 10, 13, 20, 21, 22, 24, and 27 of SEQ ID NO:1 comprise ribo/deoxyribo nucleobases modified with a 2-fluoro (F) group.

In some embodiments, nucleotides corresponding to positions 4, 10, 14, 16, 17, 18, 21, 24, 28, 30, 31, 32, 35, 36, 37, 38, 39, 40, 41, 42, 43, 44, 45, 47, 48, 51, 52, 54, 55, 56, 58, 59, 60, 62, 63, 65, 66, 67, 70, 77, 78, 79, 81, 84, 88 of SEQ ID NO:2 comprise ribo/deoxyribo nucleobases modified with a 2′-fluoro (F), amino (NH2) or O-methyl (OCH3) group.

In some embodiments, nucleotides corresponding to positions 4, 10, 14, 16, 17, 18, 21, 24, 28, 30, 31, 32, 35, 36, 37, 38, 39, 40, 41, 42, 43, 44, 45, 47, 48, 51, 52, 54, 55, 56, 58, 59, 60, 62, 63, 65, 66, 67, 70, 77, 78, 79, 81, 84, 88 of SEQ ID NO:2 comprise ribo/deoxyribo nucleobases modified with a 2′-fluoro (F), group.

In some embodiments, nucleotides corresponding to positions 4, 10, 14, 16, 17, 18, 21, 24, 28, 30, 31, 32, 35, 36, 37, 38, 39, 40, 41, 42, 43, 44, 45, 47, 48, 51, 52, 54, 55, 56, 58, 59, 60, 62, 63, 65, 66, 67, 70, 77, 78, 79, 81, 84, 88 of SEQ ID NO:2 comprise ribo/deoxyribo nucleobases modified with a 2′-fluoro (F) group.

In some embodiments, a nucleic acid compound provided herein may comprise one or more deoxyribonucleotide residues. That is, a nucleic acid compound may comprise an RNA/DNA sequence as described hereinabove, and additionally one or more deoxyribonucleotide residues. In such cases, the compound may be described as a deoxyribonucleic acid compound.

Any nucleic acid compound disclosed herein may be isolated and/or substantially purified.

### Sequence Identity

The terms “identical” or percent “identity,” in the context of two or more nucleic acid or polypeptide sequences, refer to two or more sequences or subsequences that are the same or have a specified percentage of amino acid residues or nucleotides that are the same (i.e., about 60% identity, preferably 65%, 70%, 75%, 80%, 85%, 87%, 90%, 91%, 92%, 93%, 94%, 95%, 96%, 97%, 98%, 99%, or higher identity over a specified region, when compared and aligned for maximum correspondence over a comparison window or designated region) as measured using e.g. a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from about 10 to 600, usually about 50 to about 200, more usually about 100 to about 150 in which a sequence may be compared to a reference sequence of the same number of contiguous positions after the two sequences are optimally aligned.

Methods of alignment of sequences for comparison are well known in the art. Optimal alignment of sequences for comparison can be conducted in various ways known to a person of skill in the art, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, PASTA, and FASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)). Publicly available computer software may be used such as ClustalOmega (Söding, J. 2005, Bioinformatics 21, 951-960), T-coffee (Notredame et al. 2000, J. Mol. Biol. (2000) 302, 205-217), Kalign (Lassmann and Sonnhamrner 2005, BMC Bioinformatics, 6(298)) and MAFFT (Katoh and Standley 2013, Molecular Biology and Evolution, 30(4) 772-780 software. When using such software, the default parameters, e.g. for gap penalty and extension penalty, are preferably used.

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively.

In the cases where the compound moiety is an oligonucleotide, e.g. DNA or RNA, the sequence identity of the aptamer excludes the sequence of the oligonucleotide compound moiety.

Sequences

| SEQ ID NO: | DESCRIPTION | Sequence |
|---|---|---|
| 1 | A40s | CCUGUUGUUCGACAGGAGGCUCACAACAGG |
| 2 | 40L (A40s sequence | AGACAAGAAUAAACGCUCAAUGAUAGACAUUCGG |
| | underlined) | UGCUCUCUUUCAUUGACCGUUCACCUGUUGUUC |
| | | GACAGGAGGCUCACAACAGGC |
| 3 | P10 (Forward) | TAATACGACTCACTATAGGGAGACAAGAATAAACG |
| | | CTCAA |
| 4 | P20 (Reverse) | GCCTGTTGTGAGCCTCCTGTCGAA |
| 5 | Scrambled | TTCGTACCGGGTAGG |
| | (Forward) | |
| 6 | Scrambled | TGACACGTTCTATGTGCA |
| | (Reverse) | |
| 7 | A40s (Forward) | CATCCCTGTTGTICG |
| 8 | A40s (Reverse) | CAGGCCTGTTGTGAC |
| 9 | β-ACTIN fw | TGCGTGACATTAAGGAGAAG |
| 10 | β-ACTIN rv | GCTCGTAGCTCTTCTCCA |
| 11 | NANOG fw | CAAAGGCAAACAACCCACTT |
| 12 | NANOG rv | TCTGGAACCAGGTCTTCACC |
| 13 | GFAP fw | CTGCGGCTCGATCAACTCA |
| 14 | GFAP rv | TCCAGCGACTCAATCTTCCTC |
| 15 | miR-34c passenger | ACUAGGCAGUGUAGUUAGCUGAUUGC2′OMe(GG) |
| | strand sticky | CU2′OMe(A)UCU2′OMe(AGAA)U2′ |
| | | OMe(G)U2′OMe(A)C |
| 16 | miR-34c guide | AAUCACUAACCACACGGCCAGG |
| | strand | |

### Compound Moieties and Compounds

Nucleic acid compounds, e.g. ribo/deoxyribonucleic acid compounds, provided herein may comprise a therapeutic or diagnostic molecule.

The therapeutic or diagnostic molecule may form part of the nucleic acid compound provided herein, and is thus referred to as a “compound moiety”, e.g. a therapeutic moiety or an imaging moiety. Alternatively, the therapeutic or diagnostic molecule may not form part of the nucleic acid compound provided herein, including embodiments thereof, but may be independently internalised by a GSC cell upon binding of a nucleic acid compound provided herein to GSC. In this situation, the therapeutic or diagnostic molecule is referred to as a “compound.”

Thus, a nucleic acid compound provided herein (including embodiments thereof) may include a compound moiety. Where the nucleic acid compound includes a compound moiety, the compound moiety may be covalently (e.g. directly or through a covalently bonded intermediary) attached to the nucleic acid compound or the RNA/DNA sequence (see, e.g., useful reactive moieties or functional groups used for conjugate chemistries set forth above). Thus, in some embodiments, the nucleic acid compound further includes a compound moiety covalently attached to the nucleic acid compound or the RNA/DNA sequence. In embodiments, the compound moiety and the nucleic acid compound or the RNA/DNA sequence form a conjugate. In some embodiments, the compound moiety is non-covalently attached to the nucleic acid compound or the RNA/DNA sequence, e.g. via ionic bond(s), van der Waal's bond(s)/interactions, hydrogen bond(s), polar bond(s), “sticky bridges” (see e.g. Zhou J et al. Nucleic Acids Res. 2009; 37(9): 3094-3109) or combinations or mixtures thereof. The compound moiety may be attached to the nucleic acid compound or the RNA/DNA sequence via an intermediate molecule such as a modular streptavidin connector (see e.g. Chu T C et al., Nucleic Acids Res 2006, 34:e73). Where the compound moiety is encapsulated as described hereinbelow, e.g. in a nanoparticle or liposome, the encapsulation moiety may itself be attached, covalently or non-covalently, to the nucleic acid compound or RNA/DNA sequence.

In some embodiments, the compound moiety is a therapeutic moiety or an imaging moiety covalently attached to the nucleic acid compound or RNA/DNA sequence.

The term “therapeutic moiety” as provided herein is used in accordance with its plain ordinary meaning and refers to a monovalent compound having a therapeutic benefit (prevention, eradication, amelioration of the underlying disorder being treated) when given to a subject in need thereof. Therapeutic moieties as provided herein may include, without limitation, peptides, proteins, nucleic acids, nucleic acid analogs, small molecules, antibodies, enzymes, prodrugs, nanostructures, viral capsids, cytotoxic agents (e.g. toxins) including, but not limited to ricin, doxorubicin, daunorubicin, taxol, ethidium bromide, mitomycin, etoposide, teniposide, vincristine, vinblastine, colchicine, dihydroxyanthracenedione, actinomycin D, diphtheria toxin, Pseudomonas exotoxin (PE) A, PE40, abrin, and glucocorticoid. In embodiments, the therapeutic moiety is an anti-cancer agent or chemotherapeutic agent as described herein. In embodiments, the therapeutic moiety is a nucleic acid moiety, a peptide moiety or a small molecule drug moiety. In embodiments, the therapeutic moiety is a nucleic acid moiety. In embodiments, the therapeutic moiety is a peptide moiety. In embodiments, the therapeutic moiety is a small molecule drug moiety. In embodiments, the therapeutic moiety is a nuclease. In embodiments, the therapeutic moiety is an immunostimulator. In embodiments, the therapeutic moiety is a toxin. In embodiments, the therapeutic moiety is a nuclease. In embodiments, the therapeutic moiety is a zinc finger nuclease. In embodiments, the therapeutic moiety is a transcription activator-like effector nuclease. In embodiments, the therapeutic moiety is Cas9. The therapeutic moiety may be encapsulated in a nanoparticle or liposome, where the nanoparticle or liposome is attached to the nucleic acid compound or the RNA/DNA sequence.

In the cases where the compound moiety is an oligonucleotide, e.g. DNA or RNA, the sequence identity of the aptamer excludes the sequence of the oligonucleotide compound moiety.

In some embodiments, the therapeutic moiety is an activating nucleic acid moiety (a monovalent compound including an activating nucleic acid) or an antisense nucleic acid moiety (a monovalent compound including an antisense nucleic acid). An activating nucleic acid refers to a nucleic acid capable of detectably increasing the expression or activity of a given gene or protein. The activating nucleic acid can increase expression or activity 10%, 20%, 30%, 40%, 50%, 60%, 70%, 80%, 90% or more in comparison to a control in the absence of the activating nucleic acid. In certain instances, expression or activity is 1.5-fold, 2-fold, 3-fold, 4-fold, 5-fold, 10-fold or higher than the expression or activity in the absence of the activating nucleic acid. An antisense nucleic acid refers to a nucleic acid that is complementary to at least a portion of a specific target nucleic acid and is capable of reducing transcription of the target nucleic acid or reducing the translation of the target nucleic acid or altering transcript splicing. An antisense nucleic acid may be capable of detectably decreasing the expression or activity of a given gene or protein. The antisense nucleic acid can decrease expression or activity 10%, 20%, 30%, 40%, 50%, 60%, 70%, 80%, 90% or more in comparison to a control in the absence of the antisense nucleic acid.

In some embodiments, the therapeutic moiety is a miRNA moiety (a monovalent compound including a miRNA), an mRNA moiety (a monovalent compound including an mRNA), a siRNA moiety (a monovalent compound including a siRNA) or an saRNA moiety (a monovalent compound including an saRNA). In some embodiments, the therapeutic moiety is a miRNA moiety. The term “miRNA” is used in accordance with its plain ordinary meaning and refers to a small non-coding RNA molecule capable of post-transcriptionally regulating gene expression. In one embodiment, a miRNA is a nucleic acid that has substantial or complete identity to a target gene. In some embodiments, the miRNA inhibits gene expression by interacting with a complementary cellular mRNA thereby interfering with the expression of the complementary mRNA. Typically, the miRNA is at least about 15-50 nucleotides in length (e.g., each complementary sequence of the miRNA is 15-50 nucleotides in length, and the miRNA is about 15-50 base pairs in length). In other embodiments, the length is 20-30 base nucleotides, preferably about 20-25 or about 24-29 nucleotides in length, e.g., 20, 21, 22, 23, 24, 25, 26, 27, 28, 29, or 30 nucleotides in length. In some embodiments, the therapeutic moiety is a siRNA moiety as described herein. In some embodiments, the therapeutic moiety is a saRNA moiety as described herein. In embodiments, the therapeutic moiety is an anticancer agent moiety. In some embodiments, the therapeutic moiety is an mRNA moiety. In embodiments, the therapeutic moiety is a cDNA moiety.

In some cases, the nucleic acid compound or the nucleotide sequence provided herein is attached to a sense strand of a nucleotide compound moiety e.g., mRNA, miRNA, siRNA or saRNA. In some cases the nucleic acid compound or the RNA/DNA sequence is attached to an antisense strand of a nucleotide compound moiety. In some cases, the nucleic acid compound or the RNA/DNA sequence is attached to a guide strand of a nucleotide compound moiety. In some cases, the nucleic acid compound or the RNA/DNA sequence is attached to a passenger strand of a nucleotide compound moiety.

In some cases, the therapeutic moiety is a miRNA moiety. One example of a miRNA moiety could be a member of the mir-34 microRNA family (e.g. miR-34c). The miRNA miR-34c is a tumor suppressor. It is downregulated in most forms of cancers and inhibits malignant growth by repressing genes involved in processes such as proliferation, anti-apoptosis, sternness, and migration. It has been shown that miR-34c suppresses tumor growth and metastasis in nasopharyngeal carcinoma (Li et al., 2015), miR-34c targets MET in prostate cancer cells (Hagman et al 2013), miR-34c regulates Notch signaling during bone development (Bae et al., 2012), and miR-34 inhibits human p53-mutant gastric cancer tumorspheres (Ji et al., 2008).

The compound moiety provided herein may be an imaging moiety. An “imaging moiety” as provided herein is a monovalent compound detectable by spectroscopic, photochemical, biochemical, immunochemical, chemical, or other physical means. In some embodiments, the imaging moiety is covalently attached to the nucleic acid compound or the RNA/DNA sequence. Exemplary imaging moieties are without limitation ³²P, radionuclides, positron-emitting isotopes, fluorescent dyes, fluorophores, antibodies, bioluminescent molecules, chemiluminescent molecules, photoactive molecules, metals, electron-dense reagents, enzymes (e.g., as commonly used in an ELISA), magnetic contrast agents, quantum dots, nanoparticles e.g. gold nanoparticles, biotin, digoxigenin, haptens and proteins or other entities which can be made detectable, e.g., by incorporating a radiolabel into a peptide or antibody specifically reactive with a target peptide. Any method known in the art for conjugating an antibody to the moiety may be employed, e.g., using methods described in Hermanson, Bioconjugate Techniques 1996, Academic Press, Inc., San Diego. Exemplary fluorophores include fluorescein, rhodamine, GFP, coumarin, FITC, Alexa Fluor®, Cy3, Cy5, BODIPY, and cyanine dyes. Exemplary radionuclides include Fluorine-18, Gallium-68, and Copper-64. Exemplary magnetic contrast agents include gadolinium, iron oxide and iron platinum, and manganese. In some embodiments, the imaging moiety is a bioluminescent molecule. In some embodiments, the imaging moiety is a photoactive molecule. In some embodiments, the imaging moiety is a metal. In some embodiments, the imaging moiety is a nanoparticle.

The term “imaging agent” as used herein describes the imaging moieties above when they are not attached to the nucleic acid compounds described herein.

In some cases, the nucleic acid compounds described herein comprise (i) a nucleotide sequence as described herein and (ii) an additional aptamer molecule. Where the RNA/DNA sequence is an aptamer, such molecules may be described as bispecific aptamers. Preferably, the additional aptamer molecule does not target and/or bind to GSCs. In some cases, the nucleic acid compounds described herein are multivalent. In some cases, a terminus of a nucleic acid as described herein may be annealed to a terminus of an additional aptamer molecule using a complementary nucleotide linker sequence attached to each moiety (see e.g. McNamara, J. O. et al. J. Clin. Invest. 2008 118:376-386, which is hereby incorporated by reference in its entirety).

The compound moieties or compounds described herein may be conjugated to the nucleic acid compounds of the present invention by any suitable method as described herein or known in the art, see e.g. Zhu G et al., Bioconjug Chem. 2015 26(11): 2186-2197, hereby incorporated by reference in its entirety. Chemical-based linkers may employ activating reagent such as m-maleimidobenzoyl N-hydroxysuccinimide ester (MBS), 2-iminothiolane (Traut's reagent), N-succinimidyl-3-2-pyridyldithio propionate (SPDP) or may use e.g. PEGylation or avidin/biotin techniques (see e.g. Pardridge W M, Adv Drug Delivery Rev. 1999, 36:299-321; Qian Z M et al., supra, which are hereby incorporated by reference in their entirety).

### Modifications

The nucleic acid compounds described herein may contain chemical modifications, e.g. as defined herein, to enhance their functional characteristics, such as nuclease resistance or binding affinity. The modifications may be present in a nucleic acid compound, a nucleotide sequence and/or in a nucleotide-based compound moiety or compound, e.g. a saRNA, siRNA, miRNA, mRNA.

In some cases, modifications may be made to the base, sugar ring, or phosphate group of one or more nucleotides.

In some cases, the nucleic acid compounds described herein comprise one or more modified nucleobases. For example, the nucleic acid compounds may comprise one or more ribo/deoxyribo nucleobases modified with a fluoro (F), amino (NH₂) or O-methyl (OCH₃) group. In some cases, the nucleobases are modified at the 2′ position, the 3′ position, the 5′ position or the 6′ position. In some cases, the nucleic acid compounds may comprise one or more 2′-aminopyrimidines, 2′-fluoropyrimidines, 2′-O-methyl nucleotides and/or ‘locked’ nucleotides (LNA) (see e.g. Lin, Y et al., Nucleic Acids Res. 1994 22, 5229-5234 (1994); Buckman, J. et al. J. Biol. Chem. 1998 273, 20556-20567; Burmeister, P E et al., Chem. Biol. 2005 12, 25-33; Kuwahara, M. & Obika, S. Artif. DNA PNA XNA 2013 4, 39-48; Veedu, R. N. & Wengel, J. Mol. Biosyst. 2009 5,787-792). In some cases, the nucleic acid compounds comprise one or more L-form nucleic acids (see e.g. Maasch, C et al., Nucleic Acids Symp. Ser. (Oxf.) 2008 52, 61-62). Other suitable nucleic acid modifications will be apparent to those skilled in the art (see, e.g. Ni S et al., Int. J. Mol. Sci 2017 18, 1683, hereby incorporated by reference in its entirety).

In some cases, a sense and/or antisense strand of a nucleotide compound moiety, e.g., mRNA, miRNA, siRNA or saRNA, may comprise a nucleotide overhang. For example, said overhang may be a 2-nucleotide (UU) overhang. Said overhang may be on the 3′ end of one or both strands. An overhang may favour Dicer recognition of the nucleotide compound moiety.

In some cases, the nucleic acid compounds described herein comprise an inverted thymidine cap on the 3′ end, or comprise 3′-biotin. In some cases, the phosphodiester linkage in the nucleic acid compounds in replaced with methylphosphonate or phosphorothioate analog, or triazole linkages (see Ni S et al., supra).

In some cases, the nucleic acid compounds described herein comprise one or more copies of the C3 spacer phosphoramite. Spacers may be incorporated internally, e.g. between an RNA/DNA sequence and a compound moiety, or at the 5′ or 3′ end of the nucleotide sequence to attach e.g. imaging moieties.

In some cases, the nucleic acid compounds described herein comprise modifications to increase half-life and/or resist renal clearance. For example, the compounds may be modified to include cholesterol, dialkyl lipids, proteins, liposomes, organic or inorganic nanomaterials, nanoparticles, inert antibodies or polyethylene glycol (PEG) e.g. 20 kDa PEG, 40 kDa PEG. Such modifications may be at the 5′-end of the compounds. In some cases, the modification comprises a molecule with a mass above the cut-off threshold for the renal glomerulus (˜30-50 kDa). In some cases, the nucleic compounds may be formulated with pluronic gel. For examples of suitable modifications and formulations see e.g. Ni et al, supra, and Zhou and Rossi, Nat Rev Drug Disc 2017, 16 181-202; both hereby incorporated by reference in their entirety.

The nucleic acid compounds described herein may comprise a tag, such as an albumin tag. Other tags may include: poly(His) tag, chitin binding protein (CBP), maltose binding protein (MBP), Strep-tag and glutathione-S-transferase (GST). The compounds may comprise an RNA/DNA affinity tag, as described in, for example, Srisawat C and Engelke D R, Methods. 2002 26(2): 156-161 and Walker et al., Methods Mol Biol. 2008; 488: 23-40, hereby incorporated by reference in their entirety. Other suitable tags will be readily apparent to one skilled in the art.

The nucleic acid compounds described herein may comprise spacer or linker sequences between the nucleic acid portion and a compound moiety and/or tag. Suitable spacer or linker sequences will be readily apparent to one skilled in the art.

### Functional Characteristics

The nucleic acid compounds described herein may be characterised by reference to certain functional properties.

In some embodiments, any nucleic/ribonucleic/deoxyribonucleic acid compound described herein may possess one or more of the following properties, which may optionally be characterised by in vitro assay:

Binds to glioblastoma stem cells (GSCs)

Capable of binding to GSCs;

Binds specifically to GSCs;

Capable of binding specifically to GSCs;

Capable of internalising into a cell;

Capable of delivering a payload, e.g. compound moiety or compound, into a cell;

Capable of delivering a payload, e.g. compound moiety or compound, into the brain;

Has inhibitory activity;

Reduces tumour cell proliferation;

Reduces glioblastoma cell proliferation;

Inhibits stem cell sternness;

Inhibits cell growth;

Inhibits cell migration.

The binding of a nucleic acid compound to a glioblastoma stem cell can be determined by, e.g., surface plasmon resonance technology, as illustrated herein and described in Drescher et al., Methods Mol Biol. 2009; 493: 323-343.

The ability of a nucleic acid compound to be internalised by a cell can be determined using an imaging moiety conjugated to the nucleic acid compound, such as a fluorescent dye, and detecting said imaging moiety by an appropriate means. Suitable imaging methods are described herein or are well known in the art. Other methods include detecting a therapeutic moiety in brain tissue e.g. using an antibody.

The ability of a nucleic acid compound to deliver a payload into a cell can be determined by detecting the payload itself, e.g. by detection of an imaging moiety or otherwise as will be known in the art, or by detecting an effect of the successful delivery of said payload, e.g. as described herein.

The term “internalised,” “internalising,” or “internalisation” as provided herein refers to a composition (e.g., a compound, a nucleic acid compound, a therapeutic agent, an imaging agent) being drawn into the cytoplasm of a cell (e.g. after being engulfed by a cell membrane).

The term “inhibitory activity” in relation to the nucleic acid compounds of the present disclosure refers to the ability of the compound to inhibit the activity of the target cell (e.g. glioblastoma cell or GSC). Inhibitory activity includes, but is not limited to, inhibition of tumour cell proliferation, inhibition of glioblastoma cell proliferation, inhibition of stem cell sternness, inhibition of stem cell growth, inhibition of cell migration, and inhibition of stem cell migration.

The ability of a nucleic acid compound to reduce tumour cell proliferation can be determined through well-known methods in the art, such as those used in the examples of the present invention. Briefly, the current invention tested ability of a nucleic acid compound to reduce tumour cell proliferation through by using CellTiter 96H AQueous One Solution cell Proliferation Assay (Promega, Madison, Wis.) measuring the absorbance at 492 nm with Multiskan FC Microplate Photometer (Thermo Fischer Scientific) as described (Donnarumma et al., 2017).

The ability of a nucleic acid compound to reduce glioblastoma cell proliferation can also be determined through well-known methods in the art, such as those used in the examples of the present invention. Briefly, the current invention tested ability of a nucleic acid compound to reduce tumour cell proliferation through by using CellTiter 96H AQueous One Solution cell Proliferation Assay (Promega, Madison, Wis.) measuring the absorbance at 492 nm with Multiskan FC Microplate Photometer (Thermo Fischer Scientific) as described (Donnarumma et al., 2017).

The term “stem cell sternness” refers to essential characteristics of a stem cell that distinguish them from non-stem cells, such as the ability to differentiate into other types of cells, and can also divide in self-renewal to produce more of the same type of stem cells e.g. pluripotency and multipotency. Pluripotent stem cells can differentiate into most cell types. Multipotent stem cells can differentiate into a number of cell types, but only those of a closely related family of cells.

The ability of a nucleic acid compound to inhibit cell stemness refers to the ability of such compounds to inhibit the essential characteristics of a stem cell such as pluripotency, multipotency, cell division, cell growth and cell migration.

The ability of a nucleic acid compound to inhibit tumor growth can be determined through well-known methods in the art, such as those used in the examples of the present invention. Briefly, tumor growth was measured with calipers, and tumor volume calculated as follows: L*(W\hat{ }2)*3,14/6 (W is the shortest dimension and L is the longest dimension).

The ability of a nucleic acid compound to inhibit cell migration can also be determined through well-known methods in the art, such as those used in the examples of the present invention. In the current examples, migration was assayed using the transwell migration assay as described by Roscigno et al. (2017).

### Pharmaceutical Formulations

The present invention provides pharmaceutical compositions comprising the nucleic acid compounds described herein.

The nucleic acid compounds described herein may be formulated as pharmaceutical compositions or medicaments for clinical use and may comprise a pharmaceutically acceptable carrier, diluent, excipient or adjuvant. The composition may be formulated for topical, parenteral, systemic, intracavitary, intravenous, intra-arterial, intramuscular, intrathecal, intraocular, intraconjunctival, intratumoral, subcutaneous, intradermal, intrathecal, oral or transdermal routes of administration which may include injection or infusion. Suitable formulations may comprise the antigen-binding molecule in a sterile or isotonic medium. Medicaments and pharmaceutical compositions may be formulated in fluid, including gel, form. Fluid formulations may be formulated for administration by injection or infusion (e.g. via catheter) to a selected region of the human or animal body.

In some cases, the nucleic acid compound according to the present invention are formulated for injection or infusion, e.g. into a blood vessel or tumour.

Pharmaceutical compositions of the nucleic acid compounds provided herein may include compositions having a therapeutic moiety contained in a therapeutically or prophylactically effective amount, i.e., in an amount effective to achieve its intended purpose. The pharmaceutical compositions of the nucleic acid compounds provided herein may include compositions having imaging moieties contained in an effective amount, i.e., in an amount effective to achieve its intended purpose. The actual amount effective for a particular application will depend, inter alia, on the condition being treated, tested, detected, or diagnosed. When administered in methods to treat a disease, such compositions will contain an amount of active ingredient effective to achieve the desired result, e.g., modulating the activity of a target molecule, and/or reducing, eliminating, or slowing the progression of disease symptoms. Determination of a therapeutically or prophylactically effective amount of a therapeutic moiety provided herein is well within the capabilities of those skilled in the art, especially in light of the detailed disclosure herein. When administered in methods to diagnose or detect a disease, such compositions will contain an amount of an imaging moiety described herein effective to achieve the desired result, e.g., detecting the absence or presence of a target molecule, cell, or tumour in a subject. Determination of a detectable amount of an imaging moiety provided herein is well within the capabilities of those skilled in the art, especially in light of the detailed disclosure herein.

The dosage and frequency (single or multiple doses) administered to a mammal can vary depending upon a variety of factors, for example, whether the mammal suffers from another disease; the route of administration; size, age, sex, health, body weight, body mass index, and diet of the recipient; nature and extent of symptoms of the disease being treated, kind of concurrent treatment, complications from the disease being treated or other health-related problems. Other therapeutic regimens or agents can be used in conjunction with the methods and compositions described herein including embodiments thereof. Adjustment and manipulation of established dosages (e.g., frequency and duration) are well within the ability of those skilled in the art.

For any composition (e.g., the nucleic acid compounds provided, as well as combinations of an anticancer agent and the nucleic acid compound provided) described herein, the therapeutically effective amount can be initially determined from cell culture assays. Target concentrations will be those concentrations of active compound(s) that are capable of achieving the methods described herein, as measured using the methods described herein or known in the art. As is well known in the art, effective amounts for use in humans can also be determined from animal models. For example, a dose for humans can be formulated to achieve a concentration that has been found to be effective in animals. The dosage in humans can be adjusted by monitoring effectiveness and adjusting the dosage upwards or downwards, as described above. Adjusting the dose to achieve maximal efficacy in humans based on the methods described above and other methods is well within the capabilities of the ordinarily skilled artisan.

In one aspect, provided herein is a pharmaceutical composition including a nucleic acid compound as described herein, including embodiments thereof, and a pharmaceutically acceptable excipient. In some embodiments, the nucleic acid includes a compound moiety covalently attached to the nucleic acid compound or the RNA/DNA sequence. As described above, the compound moiety may be a therapeutic moiety or an imaging moiety covalently attached to the nucleic acid compound or the RNA/DNA sequence.

In some aspects, the pharmaceutical composition includes a nucleic acid compound as provided herein, including embodiments thereof, and a therapeutic agent. In some embodiments, the nucleic acid compound comprises a compound moiety. In some embodiments, the nucleic acid compound and the therapeutic agent are not covalently attached. A therapeutic agent as provided herein refers to a composition (e.g. compound, drug, antagonist, inhibitor, modulator) having a therapeutic effect. In some embodiments, the therapeutic agent is an anticancer agent. In some embodiments, the pharmaceutical composition includes a pharmaceutically acceptable excipient.

In some aspects, there is provided a pharmaceutical composition comprising a nucleic acid compound as provided herein, including embodiments thereof, and a compound as described herein. That is, the composition comprises the nucleic acid compound and a compound, e.g. a therapeutic or diagnostic molecule, which does not form part of the nucleic acid compound itself. In some cases, the nucleic acid compound comprises a compound moiety. In some cases, the pharmaceutical composition additionally comprises a therapeutic agent.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavours, salt solutions (such as Ringer's solution), alcohols, oils, gelatins, carbohydrates such as lactose, amylose or starch, fatty acid esters, hydroxymethycellulose, polyvinyl pyrrolidine, and colours, and the like. Such preparations can be sterilized and, if desired, mixed with auxiliary agents such as lubricants, preservatives, stabilizers, wetting agents, emulsifiers, salts for influencing osmotic pressure, buffers, colouring, and/or aromatic substances and the like that do not deleteriously react with the compounds of the invention. One of skill in the art will recognize that other pharmaceutical excipients are useful in the present invention.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

The term “composition” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The pharmaceutical composition is optionally in unit dosage form. In such form the preparation is subdivided into unit doses containing appropriate quantities of the active component. The unit dosage form can be a packaged composition, the package containing discrete quantities of composition, such as packeted tablets, capsules, and powders in vials or ampoules. Also, the unit dosage form can be a capsule, tablet, cachet, or lozenge itself, or it can be the appropriate number of any of these in packaged form. The unit dosage form can be of a frozen dispersion.

### Methods of Delivery

As described above the nucleic acid compounds, e.g. ribo/deoxyribonucleic acid compounds provided herein, including embodiments thereof, may be used to deliver compound moieties or compounds (e.g., therapeutic agents or imaging agents) into a cell. Where a compound moiety (e.g., therapeutic moiety or imaging moiety) is delivered into a cell, the compound moiety may be covalently attached to the nucleic acid compound provided herein including embodiments thereof. Upon binding of the nucleic acid compound to a GSC, the compound moiety may be internalized by the cell while being covalently attached to the nucleic acid compound. Thus, in one aspect, a method of delivering a compound moiety into a cell is provided. The method includes, (i) contacting a cell with the nucleic acid compound, or composition, as provided herein including embodiments thereof and (ii) allowing the nucleic acid compound to bind to a GSC and pass into the cell thereby delivering the compound moiety into the cell.

Alternatively, where a compound is delivered into a cell, the compound (e.g., a therapeutic agent or an imaging agent) may not be covalently attached to the nucleic acid compound. Upon binding of the nucleic acid compound provided herein, including embodiments thereof, to a GSC, the nucleic acid compound and the compound provided may be internalized by the cell without being covalently attached to each other. Thus, in another aspect, a method of delivering a compound into a cell is provided. The method includes (i) contacting a cell with a compound and the nucleic acid compound, or composition, as provided herein including embodiments thereof and (ii) allowing the nucleic acid compound to bind to a GSC and the compound to pass into the cell thereby delivering the compound into the cell. In embodiments, the compound is a therapeutic agent or imaging agent. In embodiments, the compound is non-covalently attached to the nucleic acid compound.

The methods may be performed in vitro, ex vivo, or in vivo.

### Therapeutic and Prophylactic Applications

The nucleic acid compounds, e.g. ribo/deoxyribonucleic acid compounds, and compositions provided herein find use in therapeutic and prophylactic methods.

The present invention provides nucleic acid compounds and compositions described herein for use in a method of medical treatment or prophylaxis. The invention also provides the use of nucleic acid compounds and compositions described herein in the manufacture of medicaments for treating or preventing a disease or disorder. The invention described herein also provides methods of treating or preventing a disease or disorder, comprising administering to a subject in need thereof a therapeutically or prophylactically effective amount of a nucleic acid compound or composition described herein.

As used herein, “treatment” or “treating,” or “palliating” or “ameliorating” are used interchangeably herein. These terms refer to an approach for obtaining beneficial or desired results including but not limited to therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made. Treatment includes preventing the disease, that is, causing the clinical symptoms of the disease not to develop by administration of a protective composition prior to the induction of the disease; suppressing the disease, that is, causing the clinical symptoms of the disease not to develop by administration of a protective composition after the inductive event but prior to the clinical appearance or reappearance of the disease; inhibiting the disease, that is, arresting the development of clinical symptoms by administration of a protective composition after their initial appearance; preventing re-occurring of the disease and/or relieving the disease, that is, causing the regression of clinical symptoms by administration of a protective composition after their initial appearance.

The nucleic acid compounds described herein find use in the treatment or prevention of any disease/disorder which would benefit from the delivery of said compounds, and/or associated therapeutic or imaging moieties, to GSCs.

It will be appreciated that the therapeutic and prophylactic utility of the present invention extends to the treatment of any subject that would benefit from the delivery of a compound moiety or compound into a GSC, or into the brain.

Glioblastoma, or astrocytoma WHO grade IV, is the most fatal primary brain cancer found in humans, and most glioblastomas manifest rapidly de novo, without recognizable precursor lesions (Bleeker et al., 2012). The standard treatment for newly diagnosed glioblastoma patients is gross total removal, if possible, followed by the combination of the alkylating cytostatic drug temozolomide (TMZ) and RT (Stupp et al., 2005, Stupp et al., 2009). Clinically, gliomas are divided into four grades and the most aggressive of these, grade IV astrocytoma or glioblastoma, is also the most common in humans (Kleihues and Cavanee, 2000).

A sub-population of cells within glioblastomas with stem-like properties may be the source of tumors these cells (GSCs) are highly resistant to current cancer treatments. These cancer therapies, while killing the majority of tumor cells, ultimately fail in glioblastoma treatment because they do not eliminate GSCs, which survive to regenerate new tumors (Rodrigues Alves et al., 2011). These GSCs reside in a niche around arterioles, protecting these cells against therapy by maintaining a relatively hypoxic environment (Hira et al., 2015).

In some embodiments, the methods of treatment described herein comprise administering to a subject in need thereof a therapeutically or prophylactically effective amount of a nucleic acid compound or composition as described herein, wherein the nucleic acid compound comprises an anticancer therapeutic moiety. In some embodiments, the methods of treatment further comprise administering to a subject in need thereof an effective amount of an anticancer agent.

In some cases, the methods of treatment described herein comprise inducing or inhibiting autophagy, for example through the activation or inhibition of Beclinl. See e.g. Jin and White, Autophagy 2007; 3(1):28-31; Rosenfeldt and Ryan, Expert Rev Mol Med. 2009; 11:e36; and Mah and Ryan, Cold Spring Harb Perspect Biol. 2012; 4(1): a008821, all hereby incorporated by reference in their entirety. In some cases, the methods of treatment described herein comprise inducing or inhibiting the activity of nuclear factor kappa-light-chain-enhancer of activated B cells (NF-κB).

Where combination treatments are contemplated, it is not intended that the agents (i.e. nucleic acid compounds) described herein be limited by the particular nature of the combination. For example, the agents described herein may be administered in combination as simple mixtures as well as chemical hybrids. An example of the latter is where the agent is covalently linked to a targeting carrier or to an active pharmaceutical. Covalent binding can be accomplished in many ways, such as, though not limited to, the use of a commercially available cross-linking agent.

An “effective amount” is an amount sufficient to accomplish a stated purpose (e.g. achieve the effect for which it is administered, treat a disease, reduce enzyme activity, reduce one or more symptoms of a disease or condition, reduce viral replication in a cell). An example of an “effective amount” is an amount sufficient to contribute to the treatment, prevention, or reduction of a symptom or symptoms of a disease, which could also be referred to as a “therapeutically effective amount”. A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme or protein relative to the absence of the antagonist. A “function disrupting amount,” as used herein, refers to the amount of antagonist required to disrupt the function of an enzyme or protein relative to the absence of the antagonist. Guidance can be found in the literature for appropriate dosages for given classes of pharmaceutical products. For example, for the given parameter, an effective amount will show an increase or decrease of at least 5%, 10%, 15%, 20%, 25%, 40%, 50%, 60%, 75%, 80%, 90%, or at least 100%. Efficacy can also be expressed as “-fold” increase or decrease. For example, a therapeutically effective amount can have at least a 1.2-fold, 1.5-fold, 2-fold, 5-fold, or more effect over a control. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Patient”, “subject” or “subject in need thereof” refers to a living organism suffering from or prone to a disease or condition that can be treated by using the methods provided herein. The term does not necessarily indicate that the subject has been diagnosed with a particular disease, but typically refers to an individual under medical supervision. Non-limiting examples include humans, other mammals, bovines, rats, mice, dogs, monkeys, goat, sheep, cows, deer, and other non-mammalian animals. In some embodiments, a patient is human.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present invention can be delivered transdermally, by a topical route, formulated as applicator sticks, solutions, suspensions, emulsions, gels, creams, ointments, pastes, jellies, paints, powders, and aerosols.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

### Methods of Detecting a Cell

The nucleic acid compositions, e.g. ribo/deoxyribonucleic acid compounds, provided herein may also be used for the delivery of compounds and compound moieties to a GSC. As described above, the compounds and compound moieties delivered may be imaging agents useful for cell detections. Thus, in one aspect, a method of detecting a cell is provided. The method includes (i) contacting a cell with the nucleic acid compound, or composition, as provided herein including embodiments thereof, wherein the nucleic acid compound further includes an imaging moiety, (ii) the nucleic acid compound, or composition, is allowed to bind to a cell and pass into the cell, (iii) the imaging moiety is detected thereby detecting the cell.

In another aspect, a method of detecting a cell is provided. The method includes (i) contacting a cell with an imaging agent and the nucleic acid compound, or composition, as provided herein including embodiments thereof, (ii) the nucleic acid compound, or composition, is allowed to bind to a cell and the imaging agent is allowed to pass into the cell, (iii) the imaging agent is detected thereby detecting the cell.

The methods may be performed in vitro, ex vivo, or in vivo.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

Aspects and embodiments of the present invention will now be discussed with reference to the accompanying figures. Further aspects and embodiments will be apparent to those skilled in the art.

### EXAMPLES

In order to isolate a GSC-specific aptamer, we took advantage of a panel of primary cultures of GSCs isolated from patients' tumors. These cells are typically propagated as tumorspheres, but can be induced to differentiate toward an adherent like phenotype by growing them in serum-containing medium on a matrigel substrate in the absence of growth factors (Ricci-Vitiani et al., 2010, Pallini et al., 2008). For initial selection, we used two cell lines from patients with different GBM subtypes as a complex target to generate a panel of aptamers distinguishing stem-like cells from adherent, differentiated counterparts. We characterized one aptamer (in long and truncated forms) that binds to a target that is absent or poorly represented on cells grown in adherent conditions, but that is expressed by a subset of GSCs grown as tumorspheres. The aptamers revealed to be functionally active on GSCs, inhibiting cell growth and migration, and also had the property of being quickly internalized into GSCs, so represent a selective vehicle for therapeutics. Our results provide a blueprint for the isolation of highly selective reagents as imaging tools and cytotoxic adjuvants for the clinical management of GBM.

We describe aptamers namely, 40 L (SEQ ID NO:2) and A40s (SEQ ID NO:1), which bind GSCs. These aptamers were generated using a cell-SELEX approach on human primary GSCs. The aptamers were selective for human GSCs, were able to inhibit sternness, cell growth, and migration, and strongly reduced tumor proliferation in vivo. Moreover, 40 L (SEQ ID NO:2) and A40s (SEQ ID NO:1) were rapidly internalized upon target binding and, therefore, may serve as selective vehicles for therapeutics. Given the role of GSCs in GBM recurrence and therapy resistance, 40 L (SEQ ID NO:2) and A40s (SEQ ID NO:1) represent innovative drug candidates for GBM.

### Example 1—Materials and Methods

### Glioblastoma Stem-Cell Isolation and Differentiation

GBM tissue samples were obtained from the Institute of Neurosurgery, School of Medicine, Universitá Cattolica, Rome, Italy after craniotomy of adult patients (as described by Pallini et al., 2008) from which, before surgery, informed consent was obtained. Mechanical dissociation of GBM tumor specimens allowed stem cell isolation, as previously described (Ricci-Vitiani et al., 2010, Pallini et al., 2008). Cells were then cultured in a serum-free medium supplemented with EGF and bFGF. Differentiation was induced by plating cells on flasks coated with BD Matrigel™ Basement Membrane Matrix (BD Biosciences) in the presence of 10% serum and absence of EGF and bFGF for 2 weeks.

### Whole-Cell SELEX

The SELEX cycle was performed essentially as described elsewhere (Fitzwater and Polisky, 1996). Given the resistance to degradation against serum nucleases provided by the fluoropyrimidine, transcription was performed in the presence of 1 mM 2′-F pyrimidines and a mutant form of T7 RNA polymerase (2.5 u/μl T7 R&DNA polymerase, Epicentre Biotechnologies, Madison (Wis.), USA) was used to improve yields. The complexity of the starting pool was roughly 10¹⁴. Before each incubation with the cells, the 2′F-Py RNAs were heated at 85° C. for 5 min, snap-cooled on ice for 2 min, and allowed to warm up to 37° C.

Selection step. To sort aptamers able to selectively bind GSCs, a selection step was performed, incubating the pool with 10⁶ GSCs cells at 37° C. for 30 min up to the 14^{th} round or for 15 min in the last two rounds of SELEX. The bound aptamers were recovered after washings (one for the first two cycles and two for the others cycles) with 5 ml of serum-free DMEM-F12.

Counter selection step. To select sequences specifically recognizing GSCs cells, counter selection against glioblastoma differentiated cells was performed before the selection step. In this case the pool was first incubated for 30 min (one time up to the 13th round and two times in the last three rounds) with 10⁶ GSCs (150-mm cell plate), and unbound sequences were recovered for the selection phase.

During the selection process, we increased the number of counter selections or of washings and decreased incubation time to progressively raise the SELEX selective pressure. The use of polyinosinic acid (Sigma-Aldrich) as competitor was introduced to minimize non-specific binding. At the end of SELEX, sequences of the pools were cloned with TOPO-TA cloning kit (Invitrogen Life Technologies) before sequencing. Afterwards, they were compared by Clustal and their structure predictions were obtained with RNAstructure or DNASIS software.

### Binding and Internalization Analysis

2×10⁵ cells were treated with 200 nM of individual aptamers (or the starting pool as a negative control) for 30 minutes at 37° C. in the presence of 100 μg/ml polyinosine used as a nonspecific competitor (Sigma-Aldrich). Following two washes with PBS, to remove unbound RNA, bound RNA was recovered by TRIzol (Life Technologies) containing 0.5 pmol/ml of a non-related aptamer used as a reference control (at each experiment, the obtained data were normalized to the reference control). The amount of bound RNAs was determined by performing RT qPCR, as reported, with the following primers for the long sequences: P10(Forward): 5′-TAATACGACTCACTATAGGGAGACAAGAATAAACGCTCAA-3′ (SEQ ID NO:3), P20 (Reverse): 5T-GCCTGTTGTGAGCCTCCTGTCGAA-3′ (SEQ ID NO:4), Scrambled aptamer and A40s were amplified respectively with the following primers Scrambled (Forward): 5T-TTCGTACCGGGTAGG-3′ (SEQ ID NO:5), Scrambled (Reverse): 5′-TGACACGTTCTATGTGCA-3′ (SEQ ID NO:6), A40s (Forward): 5′-CATCCCTGTTGTTCG-3′, A40s (Reverse) 5′-CAGGCCTGTTGTGAC-3′.To check internalization, cell surface-bound aptamers were removed by washing three times the cells before recovering with PBS/0.5 M NaCl. Internalization rate is expressed as percentage of internalized aptamer compared to total bound aptamer.

### Western Blot Analysis

After washing cells twice in ice-cold PBS, protein extracts were prepared by incubating cell pellets in JS buffer (50 mM HEPES pH 7.5 containing 150 mM NaCl, 1% glycerol, 1% Triton X100, 1.5 mM MgCl₂, 5 mM EGTA, 1 mM Na₃VO₄, and 1× protease inhibitor cocktail). Protein concentrations was determined by Bio-Rad Protein Assay reagent, and equal amounts of proteins were separated by SDS-PAGE (10% polyacrylamide gel). The separated proteins were transferred to nitrocellulose membranes (Millipore, Bedford, Mass.). Membranes were blocked for 1 h with 5% non-fat dry milk in Tris Buffered Saline (TBS) containing 0.1% Tween-20. Primary antibodies were incubated at 4° C. overnight, peroxidase-conjugated secondary antibodies were used to perform an enhanced chemiluminescence (ECL Star, Euroclone, Milan, Italy) reaction according to the manufacturer's protocol in order to identify target proteins. Primary antibodies used were: anti-β3tubulin, anti-GFAP, anti-Sox2 (Santa Cruz Biotechnologies, Mass.), anti-βactin (Sigma, Milan, Italy).

### In Vitro Limiting Dilution Assay

The assay was performed as previously described by Adamo et al. A number of 1, 5, or 10 cells per well were seeded in stem cell medium into a 96-well plate. Two weeks after seeding, the number of wells containing spheroids for each cell plating density was counted, and extreme limiting dilution analysis was performed using software available from WEHI, Australia. For clear and unambiguous understanding, the reciprocal of 95% confidence intervals for 1/(stem cell frequency) generated by ELDA software was calculated and shown in graph. Given the long period of treatment, aptamers were renewed in wells twice a week at a concentration of 100 nM.

### Cell Viability

Dissociated tumor spheres were counted and 1×10⁵ cells/point were pretreated with the aptamer or the starting pool, as a negative control, at a concentration of 400 nM. Following 72 h, cells were seeded (1×10³ cells/well in 96-well plates) and treated with the aptamer or the starting pool at 400 nM. For long treatments, aptamers were renewed two times a week at 100 nM. Cell viability was assessed by using CellTiter 96H AQueous One

Solution cell Proliferation Assay (Promega, Madison, Wis.) measuring the absorbance at 492 nm with Multiskan FC Microplate Photometer (Thermo Fischer Scientific) as described (Donnarumma et al., 2017).

### Transwell Migration Assay

Dissociated tumor spheres were counted and 1.5×10⁵ cells/point were pretreated with the aptamer or the starting pool, as a negative control, at 400 nM. Following 72 h, 1×10⁵ cells were seeded in the upper chamber of transwell (Corning, Corning, N.Y., USA) in serum-free DMEM-F12. 10% FBS was used to induce cell migration toward the lower chamber. Migrated cells were visualized 24 h after seeding by staining with 0.1% crystal violet in 25% methanol. The percentage of migrated cells was evaluated by eluting crystal violet with 1% sodium dodecyl sulphate (SDS) and reading the absorbance at 594 nm, as described (Roscigno et al., 2017). RNA extraction and real-time PCR

After treating cells with aptamers or chimera, total RNAs (miRNA and mRNA) were extracted using EuroGOLDTriFast (EuroClone, Milan, Italy) according to the manufacturer's protocol. All the RNAs were reverse transcribed as described by Iaboni et al. (2016). Therefore, reverse transcription of total mRNA was performed starting from equal amounts of total RNA/sample (500 ng) using SuperScript® III Reverse Transcriptase (Invitrogen, Milan, Italy). By contrast, reverse transcription of total miRNA was performed starting from equal amounts of total RNA/sample (500 ng) using miScript reverse Transcription Kit (Qiagen, Hilden, Germany). Quantitative analyses of GFAP, NANOG and β-ACTIN (as an internal reference) were performed by real-time PCR using specific primers (IDT, Bologna, Italy) and iQ™ SYBR Green Supermix (Bio-Rad). Quantitative analysis of miRNAs and RNU6B (as an internal reference) was performed by real-time PCR using specific primers (Qiagen) and miScript SYBR Green PCR Kit (Qiagen Hilden, Germany). All reactions were run in duplicate. To amplify genes of interest we used the following primers: β-ACTIN fw:5′-TGCGTGACATTAAGGAGAAG-3′ (SEQ ID NO:9), β-ACTIN rv:5′-GCTCGTAGCTCTTCTCCA-3′ (SEQ ID NO:10); NANOG fw:5′-CAAAGGCAAACAACCCACTT-3′ (SEQ ID NO:11), NANOG rv:5′-TCTGGAACCAGGTCTTCACC-3′ (SEQ ID NO:12); GFAP fw: 5′-CTGCGGCTCGATCAACTCA-3′ (SEQ ID NO:13); GFAP rv: TCCAGCGACTCAATCTTCCTC-3′ (SEQ ID NO:14).

### Immunofluorescence Analysis

Cells were treated with 500 nM Alexa488-A40s or Alexa488-unrelated aptamer (Scrambled) at 37° C. Subsequently, cells were washed two times with phosphate buffered saline (PBS) and forced to adhere on polylysinecoated glass coverslips for 15 minutes, then cells were fixed with 4% paraformaldehyde in PBS for 20 minutes at room temperature. Coverslips were washed three times in PBS, mounted with Invitrogen Gold antifade reagent with DAPI, and finally visualized by confocal microscopy. Images were captured at the same settings, enabling direct comparison of staining patterns.

### Aptamer-miRNA Chimera

For chimera production, we used RNAs synthesized by TriLink Biotechnologies (San Diego, Calif., USA). Below, we provide the sequences used for the chimera conjugate: miR-34c passenger strand sticky: ACUAGGCAGUGUAGUUAGCUGAUUGC2′OMe(GG)CU2′OMe(A)UCU2′ OMe(AGAA)U2′OMe(G)U2′OMe(A)C-3′ (SEQ ID NO:15); miR-34c guide strand: 5′-AAUCACUAACCACACGGCCAGG-3′ (SEQ ID NO:16). All RNAs have 2′-fluoropyrimidine. To prove that miR-34c is selectively delivered through the aptamer, the negative control was made up of the unannealed single portions of the chimera (miR-34c guide strand, miR-34c passenger strand sticky and not sticky A40s). To prepare A40s/miR-34c, 10 μM of passenger RNA strand and 10 μM of the guide strand, in the appropriate 10× binding buffer (200 mM N-2-Hydroxyethylpiperazine-N′-2-Ethanesulfonic Acid, pH 7.4, 1.5 M NaCl, 20 mM CaCl₂)) were firstly denatured at 95° C. for 15 minutes, subsequently brought at 55° C. for 10 minutes, and finally warmed up to 37° C. for 20 minutes. The annealed passenger and guide strand thereby obtained was combined with A40s sticky and kept 30 minutes at 37° C.

### In Vivo Experiments

Housed athymic CD-1 nude mice (nu/nu) in a highly controlled microbiological environment were injected subcutaneously with 2×10⁶ GSC #1-GFP on both flanks. To assess A40s' (SEQ ID NO:1) ability to inhibit tumor growth, mice were intravenously treated through the caudal vein with 1,600 pmol in 100 μl/injection of A40s (SEQ ID NO:1) or unrelated aptamer (named scrambled), three times a week for three weeks. Tumor growth was measured with calipers, and tumor volume calculated as follows: L*(W\hat{ }2)*3,14/6 (W is the shortest dimension and L is the longest dimension). Measurements were taken for 7 weeks, after which animals were sacrificed.

### Histology and Immuno-Histochemistry

Formalin-fixed xenografts were embedded in paraffin and cut into 5 μm-thick sections. Human KI67 (Antigen clone MIB-1 IR62; Dako UK Ltd.) staining was performed with an automatic Benchmark XT staining machine (Ventana Medical Systems Inc., Tucson, Ariz., USA) according to the manufacturer's procedure. KI67 nuclear staining intensity was evaluated by one expert pathologist. For H&E staining, 2.5 μm sections of all fixed samples were mounted on superfrost slides and treated using standard methodology.

### Statistical Analysis

Continuous variables are given as mean±1 standard deviation (SD) or standard error of the mean (SEM). Statistical values were defined using GraphPad Prism 6 (San Diego, Calif., USA) software, by student's t-test (two variables), or one-way ANOVA (more than two variables). P value 0.05 was considered significant for all analyses.

### Example 2—Results

### SELEX Selection

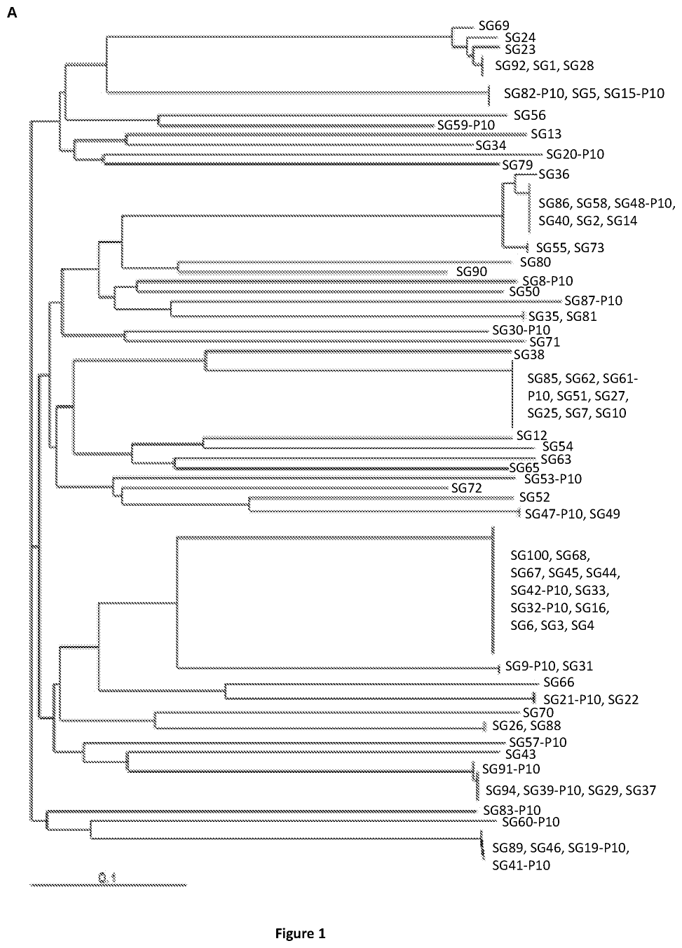

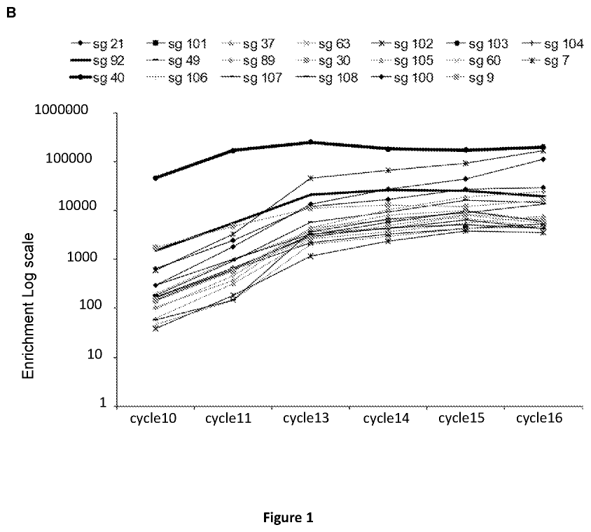

In order to isolate aptamers able to distinguish in the tumor mass the rare population of glioma cells growing as stem-like non-adherent spheres, we adopted a differential cell-SELEX approach, using primary glioma stem cell lines derived from two patients. The GSC #1 line was derived from a patient diagnosed with neural glioblastoma; the GSC #83 line was from a patient diagnosed with mesenchymal glioblastoma. Cell lines were propagated as non-adherent spheres in minimal F12 medium supplemented with cell growth factors (EGF and basic FGF), as previously described [12], and alternately used as targets in the SELEX process. Stem features were evaluated by assessing major stem cells markers. At each round, selection was preceded by one or two counterselection steps, incubating the pool with adherent GSC #1 or GSC #83 cells. For counterselections, GSC #1 or GSC #83 lines were grown as adherent cells on a matrigel substrate for two weeks in serum-containing F12 medium to induce differentiation. For the selection steps, spheres were dissociated and then incubated with the aptamer pool. Upon sixteen SELEX rounds, the final pool was cloned and 100 clones were sequenced and aligned for homology within their variable core region (FIG. 1a). Four families dominated the pool, together covering approximately 30% of sequences. To validate the information obtained by clustering, the enriched pools from rounds 10, 11, 13, 14, 15, and 16 were sequenced by high throughput sequencing (HTS) (FIG. 1b). Based on the advantages provided by each technique, the information obtained by coupling the two sequencing approaches would provide a reliable way to identify the most promising sequences. Indeed, as shown, the most enriched sequences identified by HTS belong to the four large clusters identified by conventional Sanger sequencing, on which we have focused our further analyses.

### Binding Assay

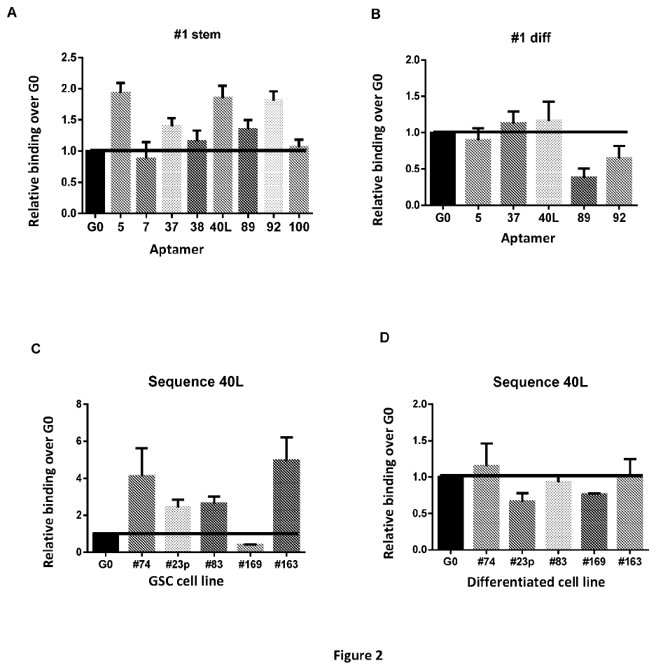

Given the good correlation between the two sequencing approaches, we determined the sequences that preferentially bound to GSCs tumor spheres as compared to adherent cells induced to differentiate. To this end, we used RT-qPCR to analyze binding at 200 nM on GSC #1 cells, the line used for the majority of selection rounds. Analysis was first performed for those aptamers that belong to the major clusters and that were rapidly enriched through the last six SELEX rounds, i.e. aptamers 5, 7, 37, 38, 40 L, 89, 92, and 100 (FIG. 2a). Sequences 5, 37, 40, 89, and 92, showing binding to GSC #1 stem cells over the untreated pool (GO), specifically distinguished growing cells, as shown by poor binding to the differentiated counterpart (FIG. 2b). We then focused on one aptamer, 40 L (SEQ ID NO:2), that was the most rapidly enriched during the SELEX rounds (at round 10). We validated the binding of 40 L (SEQ ID NO:2) on a panel of five different primary stem-like cell lines (#74; #23p; #83; #169; #163). As shown in FIG. 2C, 40 L bound to almost all the stem-like cell lines analyzed. Moreover, it showed no detectable binding for the differentiated counterparts of any of the cell lines (FIG. 2D). We also tested 40 L (SEQ ID NO:2) binding to the stem-like cells obtained from U251MG and U87MG GBM cell lines. 40 L (SEQ ID NO:2) bound to U251MG-derived stem-like cells, but not to the adherent counterpart; the aptamer did not bind U87MG stem-like cells. Given this good specificity of 40 L (SEQ ID NO:2), we restricted our further functional analyses to this sequence.

### Functional Effects of 40 L—

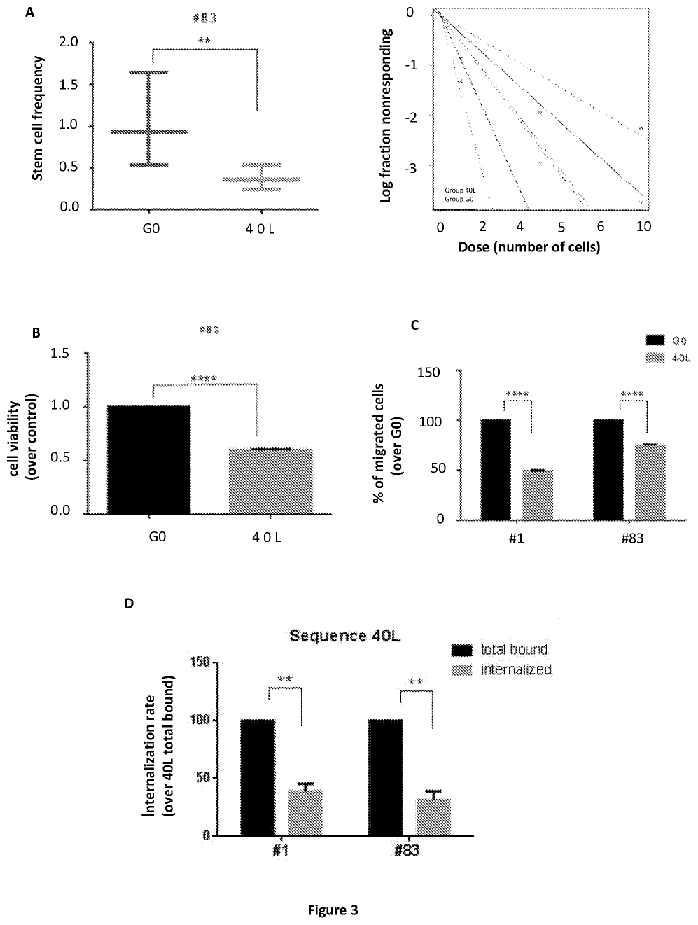

To determine the functional effects of aptamer binding to GSCs, we performed limiting dilution assay (LDA) on GSC #83 primary stem cells from a highly aggressive mesenchymal type. Data were analyzed using ELDA (Extreme Limiting Dilution Analysis) software (Hu et al., 2009). Cells treated with 40 L (SEQ ID NO:2) for 2 weeks showed approximately a 50% reduction in the estimated stem cell frequency compared to GO, used as control (FIG. 3A). Further, as determined by MTT assay, 40 L (SEQ ID NO:2) inhibited cell viability by about 50% at 6 days of treatment (FIG. 3B). We also assessed stem cell/differentiation marker expressions upon 40 L (SEQ ID NO:2) incubation. We found that 40 L (SEQ ID NO:2) induced downregulation of the stem cell-specific transcriptional factor Nanog and upregulation of the astrocyte differentiation marker GFAP (FIG. S3A). We then compared the effects of 40 L (SEQ ID NO:2) on GSC #1 and GSC #83 cell migration. With a Boyden-chamber cell migration assay, we found that 40 L (SEQ ID NO:2) interfered with both cell lines' ability to migrate toward 10% FBS, used as the chemoattractant (FIG. 3C). As previously reported, aptamer sequences for transmembrane cell surface receptors can be internalized in a receptor-mediated manner (Lao et al., 2015, Zhang et al., 2011). We thus determined if treating GSC #1 cells with 40 L (SEQ ID NO:2) would result in rapid internalization. To this end, upon 30 min of binding, we washed cells with 0.5M NaCl in PBS to remove aptamers exposed on the cell surface, before RNA extraction. As determined by RT-qPCR, approximately 40% of total bound 40 L was not affected by the NaCl wash, indicative of intracellular uptake (FIG. 3D). Taken together, these results indicate that once bound to GSCs, 40 L (SEQ ID NO:2) elicits an intrinsic biological activity, suggesting that it could be used to target stem cell phenotypes.

### Truncation of the Aptamer Sequence

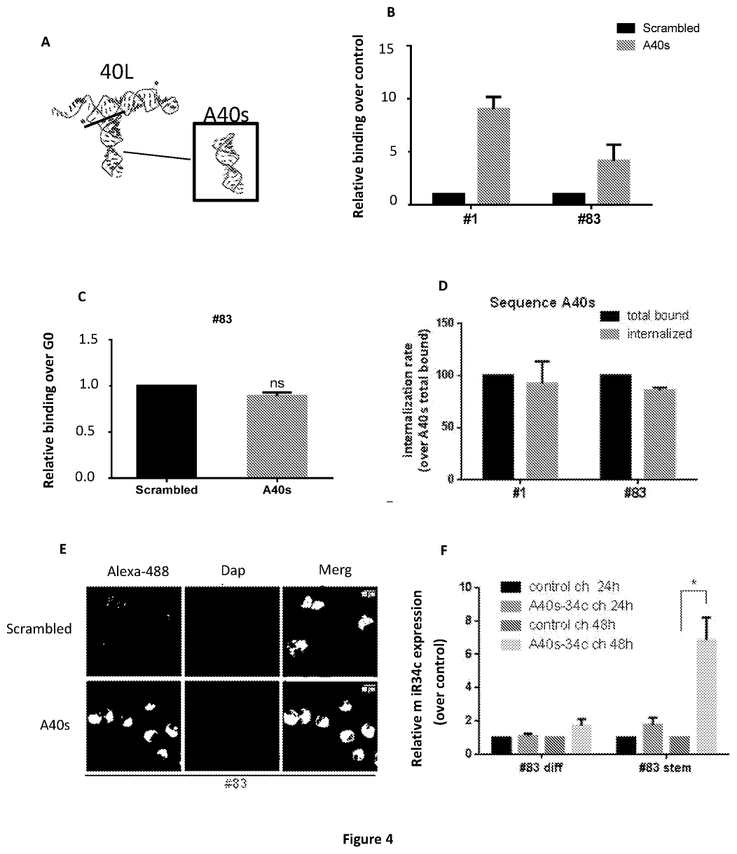

Because of its internalization by target cells, 40 L (SEQ ID NO:2) represents a good candidate for selective delivery of therapeutic molecules into GSCs. Therefore, we designed a shortened aptamer preserving the portions responsible for the binding and functional properties of the full-length aptamer. To this end, we utilized nucleic acid 2D structure prediction tools (RNA structure and DNAasis) to design and then synthesize a 30 bp sequence from base 58 to 87 (FIG. 4A), which we called A40s (SEQ ID NO:1). A40s was then tested for its ability to bind to GSCs. As shown in FIG. 4B, A40s bound to both GSC #1 and #83 cell lines, but not to the GSCs #83 grown in adherent, differentiated conditions (FIG. 4C). We then assessed the ability of A40s (SEQ ID NO:1) to internalize into GSCs. As shown in FIG. 4D, upon 30 min of treatment, almost 100% of A40s (SEQ ID NO:1) were internalized into the cells. A40s (SEQ ID NO:1) binding to GSCs was also confirmed through immunofluorescence assay. As shown in FIG. 4D, Alexa488-labeled A40s bound to and was internalized by GSC #83 more than a scrambled Alexa488-labeled aptamer. These results allow the identification of a shortened version of 40 L aptamer that preserves the binding properties of the long sequence and enables an effective reduction of the chemical synthesis cost.

### Design of an A40s-miRNA Conjugate

It is well established that aptamers function as highly selective vehicles for therapeutic substances, such as ncRNAs (Catuogno et al., 2016, Iaboni et al., 2016) to a specific target cell. Given the very rapid internalization of A40s, we tested its ability to function as a vehicle targeting GBM cells. To this end, we used sticky-end annealing to generate a molecular chimera (termed A40s-miR-34c), consisting of a duplex miRNA cargo and A40s aptamer as carrier. We fused the passenger strand of miR-34c to A40s by the means of complementary sticky ends elongated at the aptamer's and passenger strand's 3′ termini. Finally, we annealed the guide strand of the miRNA to the template. We verified the correct annealing of the conjugate by non-denaturating gel electrophoresis analysis (data not shown). Treatment with A40s-miR34c increased miR34c levels, as assessed by qRT-PCR, in GSCs but not in differentiated cells (FIG. 4F). This finding demonstrates that A40s may function as a selective carrier for GSC targeting.

### Functional Aspects of the Short Aptamer

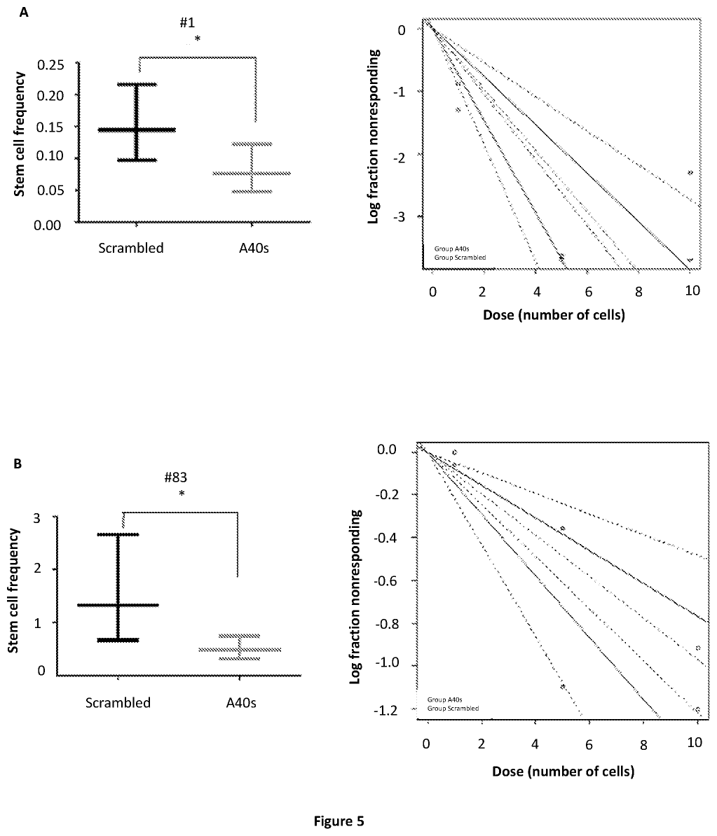

Next, we tested whether the truncated aptamer preserves the functional properties of the long sequence, evaluating the efficacy of A40s (SEQ ID NO:1) to reduce colony formation with a limiting dilution assay. We found that like the long aptamer, A40s (SEQ ID NO:1) reduced stem cell frequency about 50% in GSC #1 and #83 cell lines (FIG. 5A, B). We also assessed stem cell/differentiation marker expression upon A40s (SEQ ID NO:1) incubation: A40s (SEQ ID NO:1) induced downregulation of Nanog and upregulation of GFAP.

### Serum Stability and In Vivo Functional Aspects of A40s

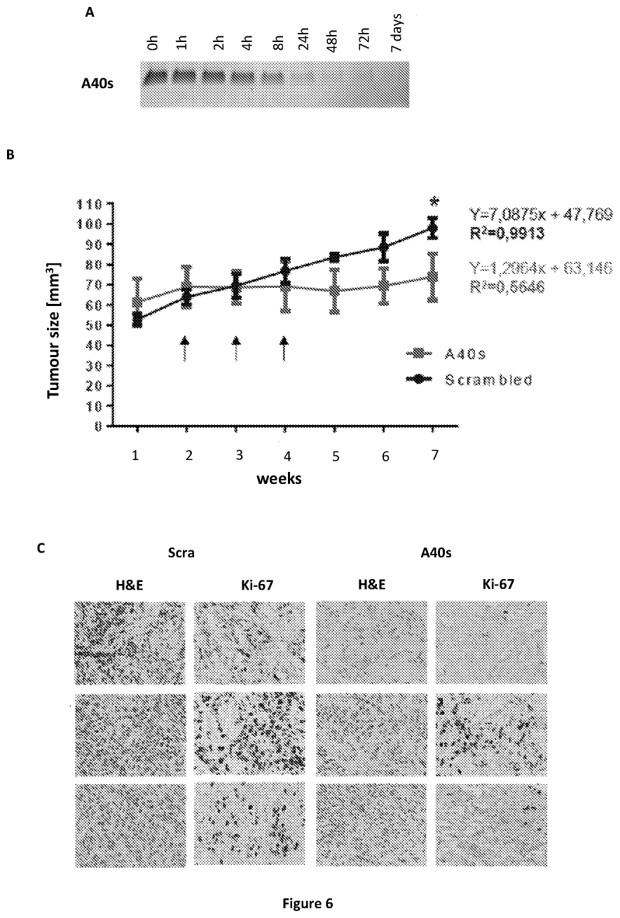

An important feature for clinical translation of new therapeutics is in vivo stability. Therefore, we evaluated the stability of A40s (SEQ ID NO:1), incubating the aptamer in human serum for up to one week. Serum RNA samples were recovered at different time-points and analyzed by non-denaturing polyacrylamide gel electrophoresis (FIG. 6A). The aptamer was found to have good stability, remaining stable in 90% serum for up to 8 hours, before being gradually degraded. We then assessed in vivo effects of A40s (SEQ ID NO:1) on subcutaneous GSC #1 xenografts. To this end, mice bearing tumors were treated with intravenous injections (1600 picomoles/injection) of A40s or scrambled control aptamer. As shown in FIG. 6b, A40s induced a strong reduction in tumor growth, affecting tumor size. This was further confirmed by histological analysis, showing decreased positivity for the proliferation marker Ki-67 (FIG. 6C). Taken together, these results indicate that A40s hampers tumor formation and has an important therapeutic potential.

### Example 3—Discussion

Glioblastoma is the most common primary brain tumor of adulthood: it is the most aggressive form of glioma, corresponding to grade IV based on WHO classification (Louis et al., 2016, Urbanska et al., 2014). Given the high capacity to invade normal brain tissue, GBM is still particularly difficult to be completely removed surgically. Despite many studies aimed at improving treatment efficacy, overall survival has not increased in a significant way over recent years. Poor prognosis is mainly caused by the almost universal recurrence of GBM within 6-9 months from treatment. GBM is a heterogeneous tumor consisting of differentiated cells and a small population of cancer stem cells (Pallini et al., 2008, Singh et al., 2003). GSCs are responsible for tumor initiation, growth, and recurrence and, thus, represent an ideal target to increase the overall survival of GBM patients.

In the present work, we addressed GSC targeting, using a nucleic acid-based aptamer. Indeed, aptamers are excellent candidates for their cell-specific recognition ability and other characteristics (e.g. short development and synthesis time, low size and cost, ease of modification, good tissue penetration, and high affinity and specificity) and represent a new class of therapeutic, diagnostic, and delivery molecules comparable, or even better than, monoclonal antibodies (Jayasena et al., 1999).

By developing an innovative cell-based selection strategy, using primary patient-derived GSCs, we identified several sequences able to effectively discriminate stem cells from their differentiated counterparts. Among the identified aptamers, we characterized in depth a sequence (40 L: SEQ ID NO:2) that showed high selectivity for GSCs isolated from different patients. Of note, 40 L exhibits functional activity on target cells: indeed, it was able to reduce sternness, cell viability, and migration, and thus has a role as a sternness regulator. Given that long RNA sequences (>60-70 nt) have high manufacturing costs, in order to improve the potential use of this aptamer as a therapeutic molecule we optimized it by identifying a shorter form (30-mer A40s: SEQ ID NO:1) able to bind GSCs like the longer 40 L. We found that, similar to 40 L, A40s discriminated between GSCs and differentiated glioma cells and, moreover, it remained functionally active on sternness. Most importantly, A40s is effective in inhibiting tumor growth in vivo in GSC-derived xenografts.

One emerging application of aptamers is as delivery tools. Here we demonstrate that 40 L and A40s show a high internalization rate in GCS cells and, thus, may be used specific carriers as well as. This was confirmed by the ability of A40s to specifically deliver miR-34c to a stem population and not to differentiated cells. Recently we reported the use of GL21.T and Gint4.T aptamers as carriers for miR-137 and antimiR-10b to target GSCs. GL21.T and Gint4.T bind Axl and PDGFRβ20, two tyrosine kinase receptors commonly expressed on GBM cells (Esposito et al., Molecular Therapy Vol. 22, Issue 6 p 1151-1163, June 2014). That paper provided a strictly defined approach to GSC targeting, identifying an aptamer that specifically targets this cell population.

The isolation of aptamers targeting GSCs has been previously described Kim Y et al. (2013). The authors described the selection of a pool of DNA sequences binding to a stem cell population. However, they did not describe any functional properties, which, in contrast, we have done for A40s. To our knowledge ours is the first description of an aptamer sequence that combines specific recognition of a stem cell population with an important functional inhibitory activity. Being a 2′-F-modified RNA, our sequence also shows an improved stability for in vivo use.

An important impediment for therapeutic compounds in GBM is the presence of the BBB, which limits the passage of large molecules to the tumor. The ability of A40s (SEQ ID NO:1) to successfully penetrate intracranially has not been investigated. Nevertheless, recent evidence supports the ability of aptamers to cross the BBB (Cheng et al., 2013, Esposito et al., 2016), and several strategies have been developed to transport therapeutics across the BBB (Abbott and Romero, 1996, Azad et al., 2015) that could be easily combined with aptamers (Monaco et al., 2017).

A40s (SEQ ID NO:1) is a good candidate for GSCs targeting and shows potential applicability as a diagnostic and a therapeutic tool. The delivery properties of the aptamer further enhance its potentiality, opening the additional possibility to develop bifunctional conjugates for effective, combined GBM therapy. Our study represents proof of principle for the development of a novel tool to target the GSC population.

### REFERENCES

A number of publications are cited above in order to more fully describe and disclose the invention and the state of the art to which the invention pertains. Full citations for these references are provided below. The entirety of each of these references is incorporated herein.
1. Louis D N, Perry A, Reifenberger G, et al. The 2016 World Health Organization Classification of Tumors of the Central Nervous System: a summary. Actaneuropathologica. 2016; 131(6):803-820.
2. Sant M, Minicozzi P, Lagorio S, et al. Survival of European patients with central nervous system tumors. International journal of cancer. 2012; 131(1):173-185.
3. Urbanska K, Sokolowska J, Szmidt M, Sysa P. Glioblastoma multiforme—an overview. Contemporary oncology. 2014; 18(5):307-312.
4. Bao S, Wu Q, McLendon R E, et al. Glioma stem cells promote radioresistance by preferential activation of the DNA damage response. Nature. 2006; 444(7120):756-760.
5. Bovenberg M S, Degeling M H, Tannous B A. Advances in stem cell therapy against gliomas. Trends in molecular medicine. 2013; 19(5):281-291.
6. Wang K, Wu X, Wang J, Huang J. Cancer stem cell theory: therapeutic implications for nanomedicine. International journal of nanomedicine. 2013; 8:899-908.
7. Ellington A D, Szostak J W. In vitro selection of RNA molecules that bind specific ligands. Nature. 1990; 346(6287):818-822.
8. Cheng C, Chen Y H, Lennox K A, Behlke M A, Davidson B L. In vivo SELEX for Identification of Brain-penetrating Aptamers. Molecular therapy. Nucleic acids. 2013; 2:e67.
9. Catuogno S, Esposito C L, de Franciscis V. Developing Aptamers by Cell-Based SELEX. Methods in molecular biology. 2016; 1380:33-46.
10. Fitzwater T, Polisky B. A SELEX primer. Methods in enzymology. 1996; 267:275-301.
11. Ricci-Vitiani L, Pallini R, Biffoni M, et al. Tumour vascularization via endothelial differentiation of glioblastoma stem-like cells. Nature. 2010; 468(7325):824-828.
12. Pallini R, Ricci-Vitiani L, Banna G L, et al. Cancer stem cell analysis and clinical outcome in patients with glioblastoma multiforme. Clinical cancer research: an official journal of the American Association for Cancer Research. 2008; 14(24):8205-8212.
13. Hu Y, Smyth G K. ELDA: extreme limiting dilution analysis for comparing depleted and enriched populations in stem cell and other assays. Journal of immunological methods. 2009; 347(1-2):70-78.
14. Lao Y H, Phua K K, Leong K W. Aptamer nanomedicine for cancer therapeutics: barriers and potential for translation. ACS nano. 2015; 9(3):2235-2254.
15. Zhang Y, Hong H, Cai W. Tumor-targeted drug delivery with aptamers. Current medicinal chemistry. 2011; 18(27):4185-4194.
16. Catuogno S, Esposito C L, de Franciscis V. Aptamer-Mediated Targeted Delivery of Therapeutics: An Update. Pharmaceuticals. 2016; 9(4).
17. Iaboni M, Russo V, Fontanella R, et al. Aptamer-miRNA-212 Conjugate Sensitizes NSCLC Cells to TRAIL. Molecular therapy. Nucleic acids. 2016; 5:e289.
18. Singh S K, Clarke I D, Terasaki M, et al. Identification of a cancer stem cell in human brain tumors. Cancer research. 2003; 63(18):5821-5828.
19. Jayasena S D. Aptamers: an emerging class of molecules that rival antibodies in diagnostics. Clinical chemistry. 1999; 45(9):1628-1650.
20. Esposito C L, Nuzzo S, Kumar S A, et al. A combined microRNA-based targeted therapeutic approach to eradicate glioblastoma stem-like cells. Journal of controlled release official journal of the Controlled Release Society. 2016; 238:43-57.
21. Kim Y, Wu Q, Hamerlik P, et al. Aptamer identification of brain tumor-initiating cells. Cancer research. 2013; 73(15):4923-4936.
22. Abbott N J, Romero I A. Transporting therapeutics across the blood-brain barrier. Molecular medicine today. 1996; 2(3):106-113.
23. Azad T D, Pan J, Connolly I D, Remington A, Wilson C M, Grant G A. Therapeutic strategies to improve drug delivery across the blood-brain barrier. Neurosurgical focus. 2015; 38(3):E9.
24. Monaco I, Camorani S, Colecchia D, et al. Aptamer Functionalization of Nanosystems for Glioblastoma Targeting through the Blood-Brain Barrier. Journal of medicinal chemistry. 2017; 60(10):4510-4516.
25. Adamo A, Fiore D, De Martino F, et al. RYK promotes the sternness of glioblastoma cells via the WNT/beta-catenin pathway. Oncotarget. 2017; 8(8):13476-13487.
26. Donnarumma E, Fiore D, Nappa M, et al. Cancer-associated fibroblasts release exosomal microRNAs that dictate an aggressive phenotype in breast cancer. Oncotarget. 2017; 8(12):19592-19608.
27. Roscigno G, Puoti I, Giordano I, et al. MiR-24 induces chemotherapy resistance and hypoxic advantage in breast cancer. Oncotarget. 2017; 8(12):19507-19521.
28. Bleeker F. E., Molenaar R. J., Leenstra S. Recent advances in the molecular understanding of glioblastoma. J Neurooncol (2012) 108: 11-27.
29. Kleihues P, Cavanee W K. World Health Organization Classification of Tumors of the Nervous System. Lyon: IARC/WHO; 2000.
30. Rodrigues Alves T., Souza Lima F. R., Kahn S. A., et al. Glioblastoma cells: A heterogeneous and fatal tumor interacting with the parenchyma. Life Sciences 89 (2011) 532-539.
31. Hira, V. V. V., Ploegmakers, K. J., Grevers, F., et al. CD133+ and Nestin+Glioma Stem-Like Cells Reside Around CD31+ Arterioles in Niches that Express SDF-1α, CXCR4, Osteopontin and Cathepsin K. Journal of Histochemistry Cytochemistry. 2015. 63 (7): 481-93.
32. Zong H, Verhaak R G, Canoll P. The cellular origin for malignant glioma and prospects for clinical advancements. Expert Review of Molecular Diagnostics. 2012. 12 (4): 383-94.
33. Zong H, Parada L F, Baker S J. Cell of origin for malignant gliomas and its implication in therapeutic development. Cold Spring Harbor Perspectives in Biology. 2015. 7 (5): a020610.
34. Li Y-Q, Ren X-Y, H Q-M et al., MiR-34c suppresses tumor growth and metastasis in nasopharyngeal carcinoma by targeting MET. Cell Death & Disease. 2015. volume 6, page e1618.
35. Hagman Z, Haflidadottir B. S., Ansari M et al. The tumour suppressor miR-34c targets MET in prostate cancer cells. British Journal of Cancer. 2013, volume 109, pages 1271-1278.
36. Bae Y, Yang T, Zeng H-C et al. miRNA-34c regulates Notch signaling during bone development. Hum Mol Genet. 2012 Jul. 1; 21(13): 2991-3000.
37. Ji Q, Hao X, Meng Y et al. Restoration of tumor suppressor miR-34 inhibits human p53-mutant gastric cancer tumorspheres. BMC Cancer. 2008; 8: 266.

For standard molecular biology techniques, see Sambrook, J., Russel, D. W. Molecular Cloning, A Laboratory Manual. 3 ed. 2001, Cold Spring Harbor, N.Y.: Cold Spring Harbor Laboratory Press.

1.-29. (canceled)

30. An RNA aptamer comprising a nucleotide sequence having at least 95% sequence identity to at least 26 contiguous nucleotides of SEQ ID NO: 1, wherein said aptamer is capable of binding to a glioblastoma stem cell.

31. The RNA aptamer of claim 30, comprising the nucleotide sequence of SEQ ID NO: 1.

32. The RNA aptamer of claim 30, wherein said nucleotide sequence has at least 95% sequence identity to at least 28 contiguous nucleotides of SEQ ID NO: 2.

33. The RNA aptamer of claim 30, wherein the RNA aptamer has a length of 50 nucleotides or fewer.

34. The RNA aptamer of claim 30, wherein the RNA aptamer has a length of 30 nucleotides.

35. The RNA aptamer of claim 30, wherein the RNA aptamer comprises a 2′ modified pyrimidine.

36. The RNA aptamer of claim 35, wherein the 2′ modified pyrimidine comprises 2′-fluoro (2′ F), 2′-amino (2′-NH2) or 2′-O-methyl (2′-OCH3).

37. The RNA aptamer of claim 35, wherein the 2′ modified pyrimidine comprises 2′-fluoro (2′ F).

38. The RNA aptamer of claim 30, further comprising a compound moiety attached to said nucleotide sequence.

39. The RNA aptamer of claim 38, wherein the compound moiety is a therapeutic moiety.

40. The RNA aptamer of claim 39, wherein the therapeutic moiety comprises:
a. a micro-RNA (miRNA), messenger RNA (mRNA), small activating RNA (saRNA), antisense nucleic acid, small interfering RNA (siRNA), short hairpin RNA (shRNA), or small nucleolar RNA (SnoRNA);
b. a MEK inhibitor or tyrosine kinase inhibitor;
c. an anti-cancer agent, alkylating agent, anti-metabolites, platinum-based compound, or angiogenesis inhibitor;
d. temozolomide, capecitabine, gemcitabine, pyrimidine analog, doxorubicin, cisplatin, oxaloplatin, or carboplatin;
e. a monoclonal antibody;
f. pembrolizumab, nivolumab, cemiplimab, dostarlimab, bevacizumab, atezolizumab, avelumab, or durvalumab;
g. an EGFR-targeted therapeutic;
h. a therapeutic radionuclide;
i. ⁶⁷Cu, ⁸⁹Sr, or ⁹⁰Y;
j. a quantum dot nanoparticle; or
k. a gold nanoparticle.

41. The RNA aptamer of claim 38, wherein the compound moiety is an imaging moiety.

42. The RNA aptamer of claim 40, wherein the imaging moiety comprises:
a. a fluorophore, radionuclide, biotin, luciferase, or nanoparticle;
b. fluorescein, rhodamine, GFP, FITC, Alexa Fluor®, Cy3, CyS, BODIPY, or cyanine dye;
c. ¹¹C, ¹³N, ¹⁵O, ¹⁸F, ¹²³I, ¹²⁵I, ¹³¹I, ⁶⁴Cu, or ³²P; or
d. a quantum dot or gold nanoparticle.

43. A method of delivering a therapeutic moiety to a glioblastoma stem cell in a subject comprising administering to the subject a pharmaceutical composition comprising the therapeutic moiety conjugated to an RNA aptamer comprising a nucleotide sequence having at least 95% sequence identity to at least 26 contiguous nucleotides of SEQ ID NO: 1.

44. The method of claim 43, wherein the therapeutic moiety comprises:
a. a micro-RNA (miRNA), messenger RNA (mRNA), small activating RNA (saRNA), antisense nucleic acid, small interfering RNA (siRNA), short hairpin RNA (shRNA), or small nucleolar RNA (SnoRNA);
b. a MEK inhibitor or tyrosine kinase inhibitor;
c. an anti-cancer agent, alkylating agent, anti-metabolites, platinum-based compound, or angiogenesis inhibitor;
d. temozolomide, capecitabine, gemcitabine, pyrimidine analog, doxorubicin, cisplatin, oxaloplatin, or carboplatin;
e. a monoclonal antibody;
f. pembrolizumab, nivolumab, cemiplimab, dostarlimab, bevacizumab, atezolizumab, avelumab, or durvalumab;
g. an EGFR-targeted therapeutic;
h. a therapeutic radionuclide;
i. ⁶⁷Cu, ⁸⁹Sr, or ⁹⁰Y;
j. a quantum dot nanoparticle; or
k. a gold nanoparticle.

45. The method of claim 43, wherein the administering comprises topical, parenteral, systemic, intracavitary, intravenous, intra-arterial, intramuscular, intrathecal, intraocular, intraconjunctival, intratumoral, subcutaneous, or intradermal administration.

46. A method of detecting a glioblastoma stem cell in a sample comprising contacting the sample with an imaging moiety conjugated to an RNA aptamer comprising a nucleotide sequence having at least 95% sequence identity to at least 26 contiguous nucleotides of SEQ ID NO: 1.

47. The method of claim 46, wherein the imaging moiety comprises:
a. a fluorophore, radionuclide, biotin, luciferase, or nanoparticle;
b. fluorescein, rhodamine, GFP, FITC, Alexa Fluor®, Cy3, CyS, BODIPY, or cyanine dye;
c. ¹¹C, ¹³N, ¹⁵O, ¹⁸F, ¹²³I, ¹²⁵I, ¹³¹I, ⁶⁴Cu, or ³⁵P; or
d. a quantum dot or gold nanoparticle.

48. The method of claim 46, wherein the sample comprises blood, serum, plasma or cerebrospinal fluid.
